SEQUENCE LISTING (13 sequences)

SEQ ID NO: 1 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 2 - protein, 8 aa, Homo sapiens
GFIVSRNY

SEQ ID NO: 3 - protein, 7 aa, Homo sapiens
IYGSGRT

SEQ ID NO: 4 - protein, 10 aa, Homo sapiens
ARDADYGLDV

SEQ ID NO: 5 - protein, 7 aa, Homo sapiens
QSVSSSY

SEQ ID NO: 6 - skipped

SEQ ID NO: 7 - protein, 10 aa, Homo sapiens
QQYGSSPLYT

SEQ ID NO: 8 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKLEIK

SEQ ID NO: 9 - protein, 116 aa, Homo sapiens
EVQLVESGGGLIQPGGSLRLSCAASGFIVSRNYMNWVRQAPGKGLEWVSVIYGSGRTDSADSVKGRFTISRDNSKNTLYLQMNSLRAEDTAVYYCARDADYGLDVWGQGTTVTVSS

SEQ ID NO: 10 - protein, 107 aa, Homo sapiens [sequence omitted]

SEQ ID NO: 11 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 216 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, Homo sapiens
EVQLVESGGGLIQPGGSLRLSCAASGFIVSRNYMNWVRQAPGKGLEWVSVIYGSGRTDSADSVKGRFTISRDNSKNTLYLQMNSLRAEDTAVYYCARDADYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0050585 A1
(43) Pub. Date: Feb. 15, 2024

(54) B7-H4-TARGETED ANTIBODY-DRUG CONJUGATES FOR THE TREATMENT OF CANCER

(71) Applicant: Mersana Therapeutics, Inc., Cambridge, MA (US)

(72) Inventors: Natalie D. KEIRSTEAD, Norristown, PA (US); Ellie E. IM, Long Island City, NY (US)

(21) Appl. No.: 18/319,176

(22) Filed: May 17, 2023

Related U.S. Application Data
(60) Provisional application No. 63/377,437, filed on Sep. 28, 2022.
(60) Provisional application No. 63/342,657, filed on May 17, 2022.

(51) Int. Cl. A61K 47/68; A61P 35/00; A61P 35/04

(52) U.S. Cl. CPC A61K 47/6849; A61P 35/00; A61K 47/68031; A61P 35/04

(57) ABSTRACT

Disclosed herein are dosing regimens for a B7-H4-targeted antibody-drug conjugates for treating cancer.

### RELATED APPLICATIONS

This application claims priority to, and the benefit of U.S. Provisional Application No. 63/342,657 filed May 17, 2022, and U.S. Provisional Application No. 63/377,437 filed Sep. 28, 2022. The contents of each of these applications are hereby incorporated by reference in their entireties.

### INCORPORATION BY REFERENCE OF SEQUENCE LISTING

The Sequence Listing XML associated with this application is provided electronically in xml file format and is hereby incorporated by reference into the specification. The name of the xml file containing the Sequence Listing is MRSN_ 038_001US_SeqList_ST26.xml. The xml file is 13,132 bytes, created on May 12, 2023, and is being submitted electronically via USPTO Patent Center.

### FIELD OF THE INVENTION

This disclosure relates generally to dosing regimens for administering B7-H4-targeted antibody-drug conjugates for the treatment of cancer.

### BACKGROUND

B7-H4, also known as B7-H4, B7x, B7S1, B7-S1, and VTCN1, is a Type I transmembrane protein and is a member of the B7 superfamily of proteins that provides a co-signal in conjunction with a T-cell receptor antigenic signal. B7-H4 is a negative regulator of T-cell function and ligation of T-cells inhibits their growth, cytokine secretion and cytotoxicity. Elimination of B7-H4 in mice does not affect immune cell homeostasis and shows no signs of autoimmunity. The receptor for B7-H4 is unknown and unidentified.

Human B7-H4 has been mapped on chromosome 1 and is comprised of six exons and five introns spanning 66 kb, of which exon 6 is used for alternative splicing to generate two different transcripts. It is a 282 amino acid protein (including the amino-terminal signal sequence), of which −227 amino acids are predicted to be in the extracellular space following cleavage of the amino-terminal signal sequence. B7-H4 comprises an Ig-like V-domain, an Ig-like C domain, a transmembrane domain and a short cytoplasmic tail.

While B7-H4 expression in healthy tissues is relatively limited at the protein level, B7-H4 is consistently overexpressed in several solid tumors such as gynecological carcinomas of the breast, ovary, and endometrium. Expression of B7-H4 in tumors tends to correlate with poor prognosis. The receptor for B7-H4 is unknown, but it is believed to be expressed on T cells. B7-H4 is believed to directly inhibit T cell activity.

A wide variety of therapeutic modalities are available for the treatment of advanced cancers including radiotherapy, conventional chemotherapy with cytotoxic antitumor agents, hormone therapy (aromatase inhibitors, luteinizing-hormone releasing-hormone analogues), bisphosphonates and signal-transduction inhibitors. Unfortunately, however, many patients either respond poorly or not at all to any of these therapeutic modalities. Thus, there is a need to identify new therapeutic agents that target the biological activities of B7-H4.

Accordingly, there exists a need for therapies that target the biological activities of B7-H4.

### SUMMARY OF THE INVENTION

The disclosure provides a method of treating a subject having TNBC, HR+/HER2− breast cancer, endometrial cancer, ovarian cancer, fallopian tube cancer, or primary peritoneal cancer or solid-tumor comprising administering to the subject a B7-H4-targeted antibody-drug conjugate (XMT-1660) by infusion at a dose of between about 7.0 mg/m² to about 90 mg/m² on the first day of treatment and every three weeks (i.e. 21 day cycle) or four weeks (i.e. 28 day cycle) thereafter, wherein the B7-H4-targeted antibody-drug conjugate is a conjugate of Formula (I):

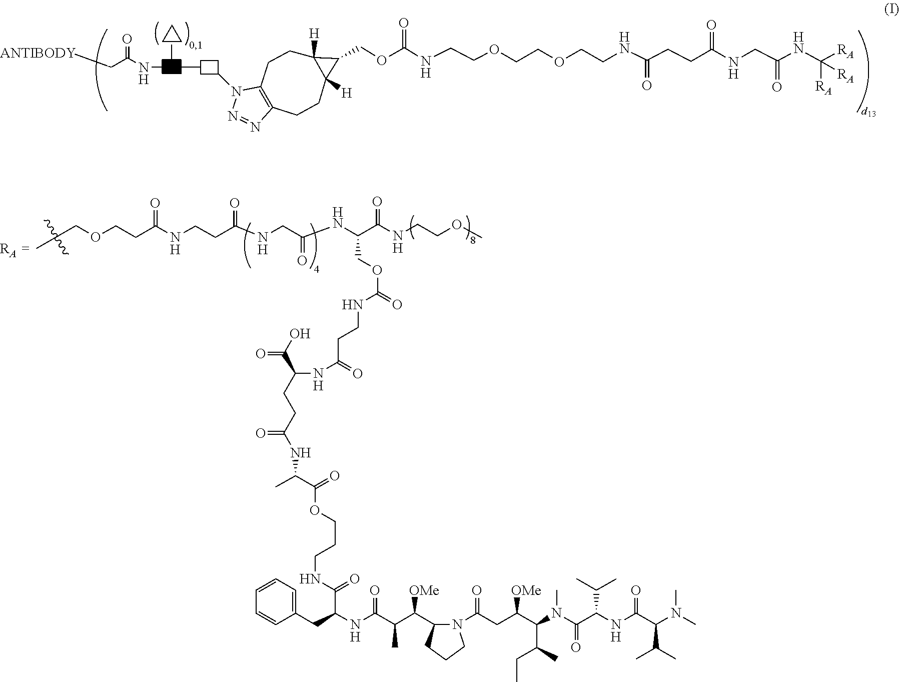

wherein
i. d₁₃ is about 2;
ii. the ANTIBODY binds B7-H4 and comprises a variable heavy chain complementarity determining region 1 (CDRH1) comprising the amino acid sequence GFIVSRNY (SEQ ID NO: 2), a variable heavy chain complementarity determining region 2 (CDRH2) comprising the amino acid sequence IYGSGRT (SEQ ID NO: 3), a variable heavy chain complementarity determining region 3 (CDRH3) comprising the amino acid sequence ARDADYGLDV (SEQ ID NO: 4), a variable light chain complementarity determining region 1 (CDRL1) comprising the amino acid sequence QSVSSSY (SEQ ID NO: 5), a variable light chain complementarity determining region 2 (CDRL2) comprising the amino acid sequence GAS (SEQ ID NO: 6), and a variable light chain complementarity determining region 3 (CDRL3) comprising the amino acid sequence QQYGSSPLYT (SEQ ID NO: 7).
iii. the drug is attached to an asparagine of the heavy chain of the antibody at position 297 when numbered in accordance with EU numbering via a linker moiety;
iv. ▪ is GlcNAc; Δ is Fuc; and □ is GalNAc.

In some embodiments, GlcNAc refers to N-acetylglucosamine (i.e., β-D-(Acetylamino)-2-deoxy-glucopyranose or N-Acetyl-D-glucosamine); Fuc refers to fucose (i.e., (2S,3R,4R,5S)-6-Methyltetrahydro-2H-pyran-2,3,4,5-tetraol or 6-Deoxy-1-galactose); and GalNAc refers to N-acetylgalactosamine (i.e., 2-(Acetylamino)-2-deoxy-D-galactose, 2-Acetamido-2-deoxy-D-galactose, N-Acetylchondrosamine, 2-Acetamido-2-deoxy-D-galactopyranose, or N-Acetyl-D-galactosamine).

In some embodiments, the subject is administered the B7-H4-targeted antibody-drug conjugate by IV infusion at a dose of about 6.7 mg/m² to about 7.7 mg/m², 13.9 mg/m² to about 14.9 mg/m², 21.1 mg/m² to about 22.1 mg/m², 28.2 mg/m² to about 29.2 mg/m², 37.6 mg/m² to about 38.6 mg/m², 50.2 mg/m² to about 51.2 mg/m², 66.9 mg/m² to about 67.9 mg/m², or 87.1 mg/m² to about 88.1 mg/m² once every 3 weeks (i.e., 21 day cycle) or every 4 weeks (i.e. 28 day cycle). In some embodiments, the subject is administered the B7-H4-targeted antibody-drug conjugate by IV infusion at a dose of about 7.2 mg/m², about 14.4 mg/m², about 21.6 mg/m², about 28.7 mg/m², about 38.1 mg/m², about 50.7 mg/m², about 67.4 mg/m² or about 87.6 mg/m² once every 3 weeks (i.e. 21 day cycle) or every 4 weeks (i.e., 28 day cycle). In some embodiments, the conjugate dose is about 7.2 mg/m². In some embodiments, the conjugate dose is about 14.4 mg/m². In some embodiments, the conjugate dose is about 21.6 mg/m². In some embodiments, the conjugate dose is about 28.7 mg/m². In some embodiments, the conjugate dose is about 38.1 mg/m². In some embodiments, the conjugate dose is about 50.7 mg/m². In some embodiments, the conjugate dose is about 67.4 mg/m². In some embodiments, the conjugate dose is about 87.6 mg/m².

In some embodiments, the subject is administered the B7-H4-targeted antibody-drug until disease progression, death, unacceptable toxicity or voluntary withdrawal—whichever comes first.

In some embodiments, the subject that has unresectable or recurrent/metastatic TNBC has received at least 2 lines of systemic therapy in locally advanced or metastatic breast cancer setting.

In some embodiments, the subject has HR+/HER2− breast cancer post-CDK4/6 inhibitor and endocrine-based therapy has received at least one line of systemic therapy which must have included CDK 4/6 inhibitor and endocrine therapy (ET), in an advanced or metastatic breast cancer setting.

In some embodiments, the subject that has endometrial cancer has received at least 1 line of systemic therapy including platinum-based chemotherapy for advanced or metastatic disease.

In some embodiments, the subject that has ovarian cancer, fallopian tube cancer, or primary peritoneal cancer has received at least 2 lines of systemic therapy for advanced or metastatic disease, which should include platinum-based chemotherapy.

In some embodiments, the cancer is B7-H4-positive.

In some embodiments, the subject is human.

Other features and advantages of the invention will be apparent from the following detailed description and claims.

### BRIEF DESCRIPTION OF THE FIGURES

FIG. 1 is a graph showing the anti-tumor efficacy of a B7-H4-targeted antibody-drug conjugate (XMT-1660) (4.68/0.150 mg/kg) and rituximab isotype control conjugate (4.60/0.150 mg/kg) (all doses are given by antibody/payload) in a patient derived endometrial cancer xenograft mouse model.

FIG. 2 is a graph showing the anti-tumor efficacy of a B7-H4-targeted antibody-drug conjugate (XMT-1660) (4.61/0.150 mg/kg) and palivizumab isotype control (4.54/0.150 mg/kg) (all doses are given by antibody/payload) in a patient derived ovarian cancer xenograft mouse model.

FIG. 3 is a graph showing the anti-tumor efficacy of a B7-H4-targeted antibody-drug conjugate (XMT-1660) (2.30/0.075 or 4.60/0.150 mg/kg, DAR 5.96) and palivizumab isotype control ADC (2.27/0.075 or 4.54/0.150 mg/kg, DAR 5.97) (all doses are given by antibody/payload) in a CTG-1692 patient derived ovarian cancer model.

FIG. 4 is a graph showing the anti-tumor efficacy of palivizumab isotype control conjugate (1.51/0.050 or 4.54/0.150 mg/kg), a B7-H4-targeted antibody-drug conjugate (XMT-1660) (1.53/0.050 or 4.60/0.150 mg/kg), anti-PD-1 (10.0/0 mg/kg), and XMT-1660+anti-PD-1 (1.53/0.050 mg/kg+10.0/0 mg/kg) (all doses are given by antibody/payload) in a mBR9013 syngeneic breast cancer mouse model.

### DETAILED DESCRIPTION

The disclosure provides methods of treating a subject having TNBC, HR+/HER2− breast cancer, endometrial cancer, ovarian cancer, fallopian tube cancer, or primary peritoneal cancer, or solid tumors thereof, comprising administering to the subject a B7-H4-targeted antibody-drug conjugate (XMT-1660) that specifically binds to the extracellular region of B7-H4. Specifically, the invention provides dosing regimens for the treatment of B7-H4 expressing cancers or solid tumors by administration as an intravenous infusion. XMT-1660 is a B7-H4-targeted antibody-drug conjugate comprising a B7-H4 modified antibody and two molecules of a fully synthetic macromolecular linker-payload bearing three copies of the microtubule inhibitor auristatin F-hydroxypropyl amide (AF-HPA). The fully synthetic macromolecular linker-payload is conjugated to the modified B7-H4-antibody in a site-specific manner enabled by glycan remodeling of the antibody at an asparagine group of the antibody. In some aspects, the asparagine is in the conserved Fc region of the antibody. In some aspects, the asparagine group is N297 according to EU numbering. The average drug-to-antibody ratio (DAR) of the B7-H4-targeted antibody-drug conjugate is approximately 6.

Patients with TNBC, HR+/HER2− breast cancer, endometrial cancer, ovarian cancer, fallopian tube cancer, or primary peritoneal cancer are intravenously administered XMT-1660 once every 3 weeks (i.e. 21 day cycle) or once every 4 weeks (i.e. 28 day cycle). Accordingly, the invention features methods of treating TNBC, HR+/HER2− breast cancer, endometrial cancer, ovarian cancer, fallopian tube cancer, or primary peritoneal cancer by administering to a subject, i.e., human, in a dose escalation study an infusion dose of XMT-1660 at 7.2 mg/m², 14.4 mg/m², 21.6 mg/m², 28.7 mg/m², 38.1 mg/m², 50.7 mg/m², 67.4 mg/m² or 87.6 mg/m² once every 3 weeks (i.e. 21 day cycle) or every 4 weeks (i.e. 28 day cycle).

In some embodiments, the subject has a B7-H4 expressing cancer. In some aspects, the subject has a B7-H4 expressing tumor.

In some embodiments the subject has been identified as having B7-H4 expression. In some embodiments, the B7-H4 expression is in the form of a B7-H4 expressing tumor. B7-H4 expression is detected by methods known in the art. For example, by immunohistochemistry (IHC) analysis, fluorescent in situ hybridization (FISH) assay or RNA expression analysis of B7-H4 transcript or other genes related to cancer measured in tumor samples. Blood-based biomarkers, which may include serum cytokines, circulating immune cells, and circulating tumor cells can also be used to determine the B7-H4 expression levels.

### B7-H4 Antibodies

Provided by the disclosure are isolated antibodies that bind to B7-H4, a Type I transmembrane protein found, for example, on the surface of antigen presenting cells (APC). The B7-H4 antibodies include, but are not limited to, humanized antibodies, chimeric antibodies, mouse antibodies, human antibodies, and antibodies comprising the heavy chain and/or light chain CDRs discussed herein.

In some embodiments, B7-H4 antibodies of the disclosure specifically bind to an epitope on the full-length human B7-H4 protein comprising the amino acid sequence:

(SEQ ID NO: 1)
MASLGQILFWSIISIIIILAGAIALIIGFGISGRHSITVTTVASAGNIG
EDGILSCTFEPDIKLSDIVIQWLKEGVLGLVHEFKEGKDELSEQDEMFR
GRTAVFADQVIVGNASLRLKNVQLTDAGTYKCYIITSKGKGNANLEYKT
GAFSMPEVNVDYNASSETLRCEAPRWFPQPTVVWASQVDQGANFSEVSN
TSFELNSENVTMKVVSVLYNVTINNTYSCMIENDIAKATGDIKVTESEI
KRRSHLQLLNSKASLCVSSFFAISWALLPLSPYLMLK.

In some embodiments, a B7-H4 antibody is a human antibody. In some embodiments, the B7-H4 antibody modulates B7-H4 activity. In some embodiments, the antibody is one that induces an Antibody-dependent cellular cytotoxicity (ADCC) response in a subject that receives the antibody. In some embodiments, the B7-H4 antibody does not inhibit T-Cell suppression activity of B7-H4. The B7-H4 antibody of this disclosure can be, for example, a full-length antibody. Alternatively, B7-H4 antibodies can be antibody fragments, such as Fab, Fab′ or Fab′ 2 fragments or single chain antibodies (e.g., scFv). In some embodiments, the antibody is an IgG1 antibody.

In some embodiments, a B7-H4 antibody comprises a heavy chain variable region and a light chain variable region. In some embodiments, a B7-H4 antibody comprises at least one heavy chain comprising a heavy chain variable region and at least a portion of a heavy chain constant region, and at least one light chain comprising a light chain variable region and at least a portion of a light chain constant region. In some embodiments, a B7-H4 antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

In some embodiments, a human B7-H4 antibody comprises one or more human constant regions. In some embodiments, the human heavy chain constant region is of an isotype selected from IgA, IgG (for example, an IgGl, IgG2, IgG3, or IgG4 , and IgD. In some embodiments, the human light chain constant region is of an isotype selected from kappa (K) and lambda (k). In some embodiments, a human antibody described herein comprises a human IgG constant region. In some embodiments, a human antibody described herein comprises a human IgG4 heavy chain constant region. In some embodiments, a human antibody described herein comprises a human IgG4 constant region and a human κ light chain.

In some embodiments, when effector function is desirable, a human B7-H4 antibody comprising a human IgGl heavy chain constant region or a human IgG3 heavy chain constant region is selected. In some embodiments, when effector function is not desirable, a human B7-H4 antibody comprising a human IgG4 or IgG2 heavy chain constant region is selected.

In some embodiments, the antibody of this disclosure is characterized by its particular functional features or properties of the antibody. For example, the antibody binds specifically to human B7-H4. Typically, the antibody of this disclosure binds to B7-H4 with high affinity, for example with a K_{D} of 1×10⁻⁷ M or less. The anti-B7-H4 antibody of this disclosure typically exhibits one or more of the following characteristics:
(a) binds to human B7-H4 with a K_{D} of 1×10⁻⁷ M or less; and/or
(b) binds to human CHO cells transfected with B7-H4 (e.g. human B7-H4).

In some embodiments, the antibody binds to human B7-H4 with a K_{D} of 5×10⁻⁸ M or less, bind to human B7-H4 with a K_{D} of 2×10⁻⁸ M or less, binds to human B7-H4 with a K_{D} of 5×10⁻⁹ M or less, binds to human B7-H4 with a K_{D} of 4×10⁻⁹ M or less, binds to human B7-H4 with a K_{D} of 3×10⁻⁹ M or less, binds to human B7-H4 with a K_{D} of 2×10⁻⁹ M or less or binds to human B7-H4 with a K_{D} of 1×10⁻⁹ M or less.

Standard assays to evaluate the binding ability of the antibody toward B7-H4 are known in the art, including for example, ELISAs, Western blots, RIAs and flow cytometry analysis. The binding kinetics (e.g., binding affinity) of the antibody also can be assessed by standard assays known in the art, such as by ELISA, Scatchard and Biacore® system analysis.

Potential therapeutic mAbs must not only bind to their target but must also be free from “developability issues” such as poor stability or high levels of aggregation. We describe guideline values for five metrics thought to be implicated in poor developability: the total length of the complementarity-determining regions (CDRs), the extent and magnitude of surface hydrophobicity, positive charge and negative charge in the CDRs, and asymmetry in the net heavy- and light-chain surface charges. The guideline cutoffs for each property were derived from the values seen in CSTs, and a flagging system is proposed to identify nonconforming candidates.

The nucleic acid and amino acid sequence of the monoclonal B7-H4 antibody of the disclosure is provided below. The complementarity determining regions (CDRs) of the heavy chain and the light chain are underlined in the amino acid sequences presented below. The amino acids encompassing the complementarity determining regions (CDR) as shown below are defined in accordance to the IMGT numbering system (See IMGT®, the international ImMunoGeneTics information system®. Available online: http://www.imgt.org/).

### B7-H4 Variable Region

In some embodiments, the B7-H4 antibody of the disclosure comprises a light chain variable region comprising or consisting of the amino acid sequence of SEQ ID NO: 8.

The V_{L} chain of B7-H4 V_{L} (SEQ ID NO:8) comprises or consists of the amino acid sequence:

(SEQ ID NO: 8)
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPLY
TFGQGTKLEIK.

The VH chain of B7-H4 (SEQ ID NO: 44) (also referred to herein as the XMT-1604 VH) comprises or consists of the amino acid sequence:

(SEQ ID NO: 9)
EVQLVESGGGLIQPGGSLRLSCAASGFIVSRNYMNWVRQAPGKGLEWVS
VIYGSGRTDSADSVKGRFTISRDNSKNTLYLQMNSLRAEDTAVYYCARD
ADYGLDVWGQGTTVTVSS.

The B7-H4 variable heavy chain is also referred to herein as the XMT-1604 variable heavy chain.

In some embodiments the B7-H4 antibody (XMT-1604) comprises a variable heavy chain complementarity determining region 1 (CDRH1) comprising the amino acid sequence GFIVSRNY (SEQ ID NO: 2), a variable heavy chain complementarity determining region 2 (CDRH2) comprising the amino acid sequence IYGSGRT (SEQ ID NO: 3), and a variable heavy chain complementarity determining region 3 (CDRH3) comprising the amino acid sequence

(SEQ ID NO: 4)
ARDADYGLDV

In some embodiments the B7-H4 antibody (XMT-1604) comprises a variable light chain complementarity determining region 1 (CDRL1) comprising the amino acid sequence QSVSSSY (SEQ ID NO: 5), a variable light chain complementarity determining region 2 (CDRL2) comprising the amino acid sequence GAS (SEQ ID NO: 6), and a variable light chain complementarity determining region 3 (CDRL3) comprising the amino acid sequence QQYGSSPLYT (SEQ ID NO: 7).

In some embodiments, the B7-H4 antibody of the disclosure has a light chain constant region comprising or consisting of the amino acid sequence:

(SEQ ID NO: 10)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

The B7-H4 light chain constant region (SEQ ID NO: 10) is also referred to herein as B7-H4 LC.

In some embodiments, the antibody of the disclosure comprises a light chain comprising or consisting of a light chain variable region amino acid sequence and a light chain constant region amino acid sequence. Antibody light chains (variable and constant regions) of the disclosure comprising or consisting of the amino acid sequence of SEQ ID NO: 12.

The B7-H4 antibody of the disclosure has a IgG1 heavy chain constant region comprising or consisting of the amino acid sequence: ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVF LFPPKPKDTLMISRTPEVTCVVVDVSHEDPEVKFNWYVDGVEVHNAKTKPREEQYNSTYR VVSVLTVLHQDWLNGKEYKCKVSNKALPAPIEKTISKAKGQPREPQVYTLPPSRDELTKNQ VSLTCLVKGFYPSDIAVEWESNGQPENNYKTTPPVLD SDGSFFLYSKLTVDKSRWQQGNVF SCSVMHEALHNHYTQKSLSLSPG (SEQ ID NO: 11). The B7-H4 IgG1 heavy chain constant region (SEQ ID NO: 11) is also referred to herein as B7-H4 HC. In some embodiments, the IgG1 heavy chain constant region comprising or consisting of SEQ ID NO: 11 further comprises one or more amino acids at the N-terminus or C-terminus. In some embodiments, the IgG1 heavy chain constant region comprises a C-terminal lysine.

In some embodiments, the antibody of the disclosure comprises a heavy chain set forth in SEQ ID NO: 13 comprising or consisting of a heavy chain variable region amino acid sequence and a heavy chain constant region amino acid sequence.

In yet another embodiment, the B7-H4 antibody of this disclosure comprises a heavy and light chain variable region comprising amino acid sequences that are homologous to the amino acid sequences of the antibody described herein and wherein the antibody retains the desired functional properties of the anti-B7-H4 antibody of this disclosure. For example, this disclosure provides an isolated monoclonal antibody or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NO: 9;
(b) the light chain variable region comprises an amino acid sequence that is at least 80% homologous to an amino acid sequence of SEQ ID NO: 8;
(c) the antibody binds to human B7-H4 with a K_{D} of 1×10⁻⁷ M or less; and
(d) the antibody binds to human CHO cells transfected with B7-H4.

In various embodiments, the antibody can be, for example, a human antibody, a humanized antibody or a chimeric antibody.

In other embodiments, the VH and/or VL amino acid sequences may be 85%, 90%, 95%, 96%, 97%, 98% or 99% homologous to the sequences set forth above. A B7-H4 antibody having VH and VL regions having high (i.e. 80% or greater) homology to the VH and VL regions of the sequences set forth above, can be obtained by mutagenesis (e.g., site-directed or PCR-mediated mutagenesis) of nucleic acid molecules encoding the amino acid sequences set forth in SEQ ID NO: 9, followed by testing of the encoded altered antibody for retained function (i.e., the functions set forth in (c) and (d) above), using the functional assays described herein.

In a preferred embodiment, the heavy chain variable region CDR2 sequence comprises an amino acid sequence SEQ ID NO: 3, and conservative modifications thereof; and the light chain variable region CDR2 sequence comprises an amino acid sequence SEQ ID NO: 6 and conservative modifications thereof. In another preferred embodiment, the heavy chain variable region CDR1 sequence comprises an amino acid sequence SEQ ID NO: 2 and conservative modifications thereof; and the light chain variable region CDR1 sequence comprises an amino acid sequence SEQ ID NO: 5 and conservative modifications thereof. In another preferred embodiment, the heavy chain variable region CDR3 sequence comprises an amino acid sequence SEQ ID NO: 4 and conservative modifications thereof; and the light chain variable region CDR3 sequence comprises an amino acid sequence SEQ ID NO: 7 and conservative modifications thereof.

In various embodiments, the antibody can be, for example, human antibody, humanized antibody or chimeric antibody.

In some embodiment, the antibody bind to the same epitope on human B7-H4 as any of the B7-H4 monoclonal antibodies of this disclosure (i.e. antibodies that have the ability to cross-compete for binding to B7-H4 with any of the monoclonal antibodies of this disclosure).

Accordingly, another embodiment of this disclosure pertains to an isolated monoclonal antibody or antigen binding portion thereof, comprising a heavy chain variable region comprising CDR1, CDR2 and CDR3 sequences comprising an amino acid sequence SEQ ID NO: 9 respectively and a light chain variable region comprising CDR1, CDR2 and CDR3 sequences comprising an amino acid sequence of SEQ ID NOs: 5, 6 and 7 respectively

Accordingly, in another embodiment, this disclosure provides isolated anti-B7-H4 monoclonal antibody or antigen binding portions thereof, comprising a heavy chain variable region comprising: (a) a VH CDR1 region comprising an amino acid sequence comprising SEQ ID NO: 2; or an amino acid sequence having one, two, three, four or five amino acid substitutions, deletions or additions as compared to SEQ ID NO: 2; (b) a VH CDR2 region comprising an amino acid sequence comprising SEQ ID NO: 3; or an amino acid sequence having one, two, three, four or five amino acid substitutions, deletions or additions as compared to SEQ ID NO: 3; (c) a VH CDR3 region comprising an amino acid sequence selected from the group consisting of SEQ ID NOs: 4 or an amino acid sequence having one, two, three, four or five amino acid substitutions, deletions or additions as compared to SEQ ID NO: 4.

In some embodiments, the antibody disclosed herein contains a heavy chain having an amino acid sequence at least 90%, 91%, 92%, 93%, 94%, 95%, 96%, 97% 98%, 99% or more identical to SEQ ID NO: 13 and a light chain having an amino acid sequence at least 90%, 91%, 92%, 93%, 94%, 95%, 96%, 97% 98%, 99% or more identical to SEQ ID NO: 12.

In some embodiments, the antibody disclosed herein contains a combination of heavy chain and light chain amino acid sequences comprising a heavy chain amino acid sequence at least 90%, 91%, 92%, 93%, 94%, 95%, 96%, 97% 98%, 99% or more identical to the amino acid sequence of SEQ ID NO: 13 and a light chain amino acid sequence at least 90%, 91%, 92%, 93%, 94%, 95%, 96%, 97% 98%, 99% or more identical to the amino acid sequence of SEQ ID NO: 12.

In some embodiments, the antibody disclosed herein comprises a heavy chain amino acid sequence at least 90%, 91%, 92%, 93%, 94%, 95%, 96%, 97% 98%, 99% or more identical to the amino acid sequence of SEQ ID NO: 13 and a light chain amino acid sequence at least 90%, 91%, 92%, 93%, 94%, 95%, 96%, 97% 98%, 99% or more identical to the amino acid sequence of SEQ ID NO: 12.

In some embodiments, the antibody disclosed herein comprises a heavy chain having an amino acid sequence consisting of SEQ ID NO: 13 and a light chain having an amino acid sequence of SEQ ID NO: 12.

In some embodiments, the antibody disclosed herein contains a combination of heavy chain and light chain amino acid sequences s of the heavy chain amino acid sequence of SEQ ID NO: 13 and the light chain amino acid sequence of SEQ ID NO: 12.

In some embodiments, the antibody disclosed herein contain the heavy chain amino acid sequence of SEQ ID NO: 13 and the light chain amino acid sequence of SEQ ID NO: 12.

The antibody disclosed herein comprises a heavy chain variable region having an amino acid sequence at least 90%, 91%, 92%, 93%, 94%, 95%, 96%, 97% 98%, 99% or more identical to SEQ ID NO: 9 and a light chain variable region having an amino acid sequence at least 90%, 91%, 92%, 93%, 94%, 95%, 96%, 97% 98%, 99% or more identical to a sequence consisting of SEQ ID NOs: 8.

In some embodiments, the three heavy chain CDRs of the antibody disclosed herein include a heavy chain complementarity determining region 1 (CDRH1) that includes an amino acid sequence at least 90%, 91%, 92%, 93%, 94%, 95%, 96%, 97% 98%, 99% or more identical to a sequence comprising SEQ ID NO: 2; a heavy chain complementarity determining region 2 (CDRH2) that includes an amino acid sequence at least 90%, 91%, 92%, 93%, 94%, 95%, 96%, 97% 98%, 99% or more identical to a sequence comprising SEQ ID NO: 3; and a heavy chain complementarity determining region 3 (CDRH3) that includes an amino acid sequence at least 90%, 91%, 92%, 93%, 94%, 95%, 96%, 97% 98%, 99% or more identical to SEQ ID Ns: 4; and a heavy chain amino acid sequence at least 90%, 91%, 92%, 93%, 94%, 95%, 96%, 97% 98%, 99% or more identical to the amino acid sequence SEQ ID NO: 13.

The three light chain CDRs of the antibody disclosed herein include a light chain complementarity determining region 1 (CDRL1) that includes an amino acid sequence at least 90%, 91%, 92%, 93%, 94%, 95%, 96%, 97% 98%, 99% or more identical to a sequence comprising SEQ ID NO. 5; a light chain complementarity determining region 2 (CDRL2) that includes an amino acid sequence at least 90%, 91%, 92%, 93%, 94%, 95%, 96%, 97% 98%, 99% or more identical to a sequence comprising SEQ ID NO: 6; and a light chain complementarity determining region 3 (CDRL3) that includes an amino acid sequence at least 90%, 91%, 92%, 93%, 94%, 95%, 96%, 97% 98%, 99% or more identical to a sequence comprising SEQ ID NO: 7.

The antibody include a combination of heavy chain CDR and light chain CDR sequences that include a CDRH1 that includes an amino acid sequence at least 90%, 91%, 92%, 93%, 94%, 95%, 96%, 97% 98%, 99% or more identical to a sequence comprising SEQ ID NO: 2; a CDRH2 that includes an amino acid sequence at least 90%, 91%, 92%, 93%, 94%, 95%, 96%, 97% 98%, 99% or more identical to a sequence comprising SEQ ID NO: 3; a CDRH3 that includes an amino acid sequence at least 90%, 91%, 92%, 93%, 94%, 95%, 96%, 97% 98%, 99% to a sequence comprising SEQ ID NO: 4; a CDRL1 that includes an amino acid sequence at least 90%, 91%, 92%, 93%, 94%, 95%, 96%, 97% 98%, 99% or more identical to a sequence selected from the group consisting of SEQ ID NO: 5; a CDRL2 that includes an amino acid sequence at least 90%, 91%, 92%, 93%, 94%, 95%, 96%, 97% 98%, 99% or more identical to a sequence of SEQ ID NO: 6; and a CDRL3 that includes an amino acid sequence at least 90%, 91%, 92%, 93%, 94%, 95%, 96%, 97% 98%, 99% or more identical to a of SEQ ID NO: 7; and a heavy chain amino acid sequence at least 90%, 91%, 92%, 93%, 94%, 95%, 96%, 97% 98%, 99% or more identical to the amino acid sequence of SEQ ID NO: 45.

The three heavy chain CDRs of the antibody disclosed herein include a CDRH1 that includes an amino acid sequence selected from the group comprising SEQ ID NO: 2 a CDRH2 that includes an amino acid sequence comprising SEQ ID NO: 3; and a CDRH3 that includes an amino acid sequence comprising SEQ ID NO: 4; and a heavy chain amino acid sequence comprising SEQ ID NO: 13.

The three light chain CDRs of the antibody disclosed herein include a CDRL1 that has an amino acid sequence of SEQ ID NO: 5; a CDRL2 that has an amino acid sequence of SEQ ID NO: 6; and a CDRL3 that has an amino acid sequence of SEQ ID NO: 7. The antibody disclosed herein include a combination of heavy chain CDR and light chain CDR sequences that include a CDHR1 that includes an amino acid sequence selected comprising SEQ ID NO: 2; a CDRH2 that includes an amino acid sequence comprising SEQ ID NO: 3; a CDRH3 that includes an amino acid sequence comprising SEQ ID NO: 4; a CDRL1 that has an amino acid sequence of SEQ ID NO: 5; a CDRL2 that has an amino acid sequence of SEQ ID NO: 6; and a CDRL3 that has an amino acid sequence of SEQ ID NO: 7; and a heavy chain amino acid sequence of SEQ ID NO: 13. The antibody disclosed herein contain a combination of heavy chain complementarity determining region and light chain complementarity determining region amino acid sequences selected from the group consisting of (i) the CDRH1 amino acid sequence of SEQ ID NO: 2, the CDRH2 amino acid sequence of SEQ ID NO: 3, the CDRH3 amino acid sequence of SEQ ID NO: 4, the CDRL1 amino acid sequence of SEQ ID NO: 5, the CDRL2 amino acid sequence of SEQ ID NO: 6, the CDRL3 amino acid sequence of SEQ ID NO: 7, and a heavy chain amino acid sequence of SEQ ID NO: 13.

In some embodiments, the antibody disclosed herein comprises a CDRH1 amino acid sequence of SEQ ID NO: 2, a CDRH2 amino acid sequence of SEQ ID NO: 3 , a CDRH3 amino acid sequence of SEQ ID NO: 4, a CDRL1 amino acid sequence of SEQ ID NO: 5, a CDRL2 amino acid sequence of SEQ ID NO: 6, a CDRL3 amino acid sequence of SEQ ID NO: 7, and a heavy chain amino acid sequence of SEQ ID NO: 13.

### Modified B7-H4 Antibodies

In some embodiments, the B7-H4 antibody is a modified B7-H4 antibody. As used herein, the term “modified B7-H4 antibody” can be used to describe a B7-H4 antibody of the disclosure having a modification including a mutation, deletion, or substitution. As used herein, the terms “modified B7-H4 antibody” can be used to describe a B7-H4 antibody having a modification to an amino acid of the antibody including covalent attachment of a chemical moiety to the amino acid side. In some embodiments, a “modified B7-H4 antibody” can have a sugar or sugar derivative moiety covalently attached to an amino acid of the antibody. In some embodiments, a “modified B7-H4 antibody” can have a sugar or sugar derivative moiety replaced by a different sugar or sugar derivative moiety.

In some embodiments of the modified B7-H4 antibody, * denotes a direct or indirect attachment to the rest of the modified B7-H4 antibody. In some embodiments, S″ is a sugar or a derivatized sugar. In some embodiments, A″ is a functional group being capable of forming a covalent bond with a functional group of a macromolecular linker-payload (i.e., drug or linker-drug moiety).

In some embodiments, the modified B7-H4 antibody, prior to conjugation, comprises a sugar-derivative moiety of *—S″—A″.

In some embodiments, the modified B7-H4 antibody comprises an asparagine group in the constant region of the antibody. The constant region of IgG antibodies is well conserved and each amino acid can be identified by EU number. See Edelman, G. M. et al., Proc. Natl. Acad. USA, 63, 78-85 (1969). In some aspects, the asparagine group is in the region 290-305 (e.g., at N297; EU numbering). Asparagine 297 is found in the conserved Fc region of the heavy chain of an IgG antibody. In some embodiments, the sugar-derivative moiety is directly or indirectly attached to the asparagine group (e.g., at N297).

In some embodiments, an asparagine of the B7-H4 antibody heavy chain constant region set forth in SEQ ID NO: 11 is modified. In some embodiments, the asparagine to be modified (N297 according to EU numbering) corresponds to position 180 (N180) of SEQ ID NO: 11. In some aspects, the macromolecular linker-payload (e.g., drug or linker-drug moiety) of the B7-H4 ADC is attached to N180 of SEQ NO: 11.

In some embodiments, an asparagine of the B7-H4 antibody heavy chain set forth in SEQ ID NO: 13 is modified. In some embodiments, the asparagine to be modified (N297 according to EU numbering) corresponds to position 296 (N296) of SEQ ID NO: 13. In some aspects, the macromolecular linker-payload (e.g., drug or linker-drug moiety) of the B7-H4 ADC is attached to N296 of SEQ NO: 13.

In some embodiments, the modified B7-H4 antibody, prior to conjugation, comprises a modified-GlcNAc moiety, *—GlcNAc—S″—A″, wherein GlcNAc is N-acetylglucosamine.

In some embodiments, the modified-GlcNAc moiety is connected to the rest of the modified B7-H4 antibody via the Cl position of the GlcNAc. In some embodiments, the modified-GlcNAc moiety further comprises a fucose.

In some embodiments, the modified-GlcNAc moiety is directly or indirectly attached to the asparagine group (e.g., at N297).

In some embodiments, the modified B7-H4 antibody is conjugated to the drug via a linker moiety (i.e., macromolecular linker-payload or linker-drug moiety) via a covalent bond formed between A″ and a functional group of the Linker-Drug moiety.

In some embodiments, the modified B7-H4 antibody of the present disclosure is obtained by a process comprising:
(a) contacting a glycoprotein (e.g., a B7-H4 antibody glycan) comprising a B7-H4 antibody and a core-GlcNAc moiety with an endoglycosidase, thereby forming an intermediate antibody comprising the antibody and a terminal-GlcNAc moiety and, optionally, the terminal-GlcNAc moiety further comprises a fucose; and
(b) contacting the intermediate antibody with a compound having the structure of P″—S″—A″, in the presence of a glycosyltransferase, thereby forming the modified B7-H4 antibody comprising the antibody and the modified-GlcNAc moiety, *—GlcNAc—S″—A″, and, optionally, the modified-GlcNAc moiety is attached to the rest of the modified B7-H4 antibody the C1 position of the GlcNAc; wherein
GlcNAc is N-acetylglucosamine;
S″ is a sugar or a derivatized sugar;
A″ is azido, keto, or alkynyl; and
P″ is uridine diphosphate (UDP), guanosine diphosphate (GDP) or cytidine diphosphate (CDP).

In some embodiments, steps (a) and (b) are conducted sequentially. In some embodiments, steps (a) and (b) are conducted concurrently.

In some embodiments, the B7-H4 antibody is an IgG1 antibody.

In some embodiments, the antibody is a full-length antibody, and the antibody glycan comprises one or more core-GlcNAc moiety.

In some embodiments, the antibody is a full-length antibody, and the antibody glycan comprises one or more core-GlcNAc moiety connected to each heavy chain of the antibody.

In some embodiments, the core-GlcNAc moiety further comprises a fucose.

In some embodiments, the antibody is a full-length antibody, and the antibody glycan comprises two or more core-GlcNAc moiety connected to the full-length antibody.

In some embodiments, the antibody is a full-length antibody, and the antibody glycan comprises two core-GlcNAc moieties connected to the full-length antibody.

In some embodiments, at least one of the two or more core-GlcNAc moieties further comprises a fucose.

In some embodiments, each of the two or more core-GlcNAc moiety further comprises a fucose.

In some embodiments, the core-GlcNAc moiety is connected to a position of the antibody, wherein the core-GlcNAc moiety does not substantially hinder the antigen-binding site of the antibody.

In some embodiments, the core-GlcNAc moiety is connected to the Fc fragment of the antibody. In some embodiments, the core-GlcNAc moiety is connected to the CH domain. In some embodiments, the core-GlcNAc moiety is connected to the Fab or Fc fragment of the antibody. In some embodiments, the core-GlcNAc moiety is connected to the antibody via an N-glycosidic bond to the amide nitrogen atom in the side chain of an asparagine amino acid of the antibody. In some embodiments, the core-GlcNAc moiety is connected to a native N-glycosylation site of the antibody.

In some embodiments, the antibody is an IgG, antibody and the core-GlcNAc moiety is connected to a native N-glycosylation site of the IgG.

In some embodiments, the antibody is an IgG antibody and the core-GlcNAc moiety is connected to a native N-glycosylation site of the IgG (e.g., the N297 N-glycosylation site of IgG, EU numbering). In some embodiments, the N297 N-glycosylation site is present in the conserved Fc region of the heavy chain of an IgG antibody at asparagine in the region 290-305 (e.g., at N297). In some embodiments, the intermediate antibody is of Formula (XXII):

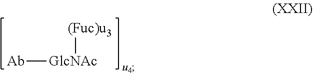

wherein: Ab is a B7-H4 antibody; GlcNAc is N-acetylglucosamine; Fuc is fucose; u₃ is 0 or 1; and u₄ is an integer ranging from is 1 to 16.

In some embodiments, u₄ is an integer ranging from 1 to 10. In some embodiments, u₄ is an integer ranging from 1 to 4. In some embodiments, u₄ is 1, 2, 3, 4, 5, 6, 7 or 8. In some embodiments, u₄ is 1, 2, 3, 4, 5 or 6. In some embodiments, u₄ is 1, 2, 3 or 4. In some embodiments, u₄ is 2 or 4. In some embodiments, u₄ is 1 or 2. In some embodiments, u₄ is 1. In some embodiments, u₄ is 2.

In some embodiments, the antibody comprises one core-GlcNAc moiety (e.g., u₄ is 1). In some embodiments, the antibody comprises two core-GlcNAc moieties (e.g., u₄ is 2).

In some embodiments, the modified B7-H4 antibody is obtained by the process outlined in Scheme 1. As shown below, contacting an intermediate antibody of Formula (XXIII) comprising one terminal-GlcNAc moiety with a compound having the structure of P″—S″—A″, in the presence of a glycosyltransferase, provides a modified B7-H4 antibody comprising one modified-GlcNAc moiety (e.g., the modified B7-H4 antibody of Formula (XXIIIa)).

In some embodiments, the modified B7-H4 antibody is obtained by contacting an intermediate antibody of Formula (XXIV) comprising two terminal-GlcNAc moieties with a compound having the structure of P″—S″—A″, in the presence of a glycosyltransferase, provides a modified B7-H4 antibody comprising two modified-GlcNAc moieties (e.g., the modified B7-H4 antibody of Formula (XXIVa)).

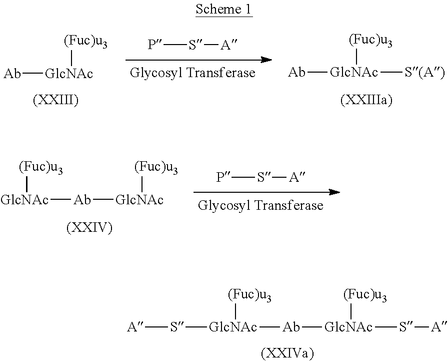

wherein u₃, Ab, S″, A″, and P″ are as defined herein.

In some embodiments, the antibody glycan to be modified in the process according to the present disclosure comprises a glycan, said glycan comprising a core-GlcNAc moiety, i.e., a GlcNAc moiety that is present at the non-reducing end of the glycan. In some embodiments, the glycan comprises one or more saccharide moieties and may be linear or branched.

In some embodiments, upon reacting with endoglycosidase, the intermediate antibody may be formed, which comprises a terminal GlcNAc moiety (e.g., the intermediate antibody of Formula (XXIII) or (XXIV)).

In some embodiments, step (a) of the process (the deglycosylation or trimming) is wherein a mixture of antibody glycoforms G2F, GIF, G0F, G2, Gl, G0, and M5 is converted into intermediate antibodies comprising a terminal GlcNAc moiety which optionally comprises a fucose (e.g., u₃ is 0 or 1).

In some embodiments the endoglycosidase is Endo S or Endo SH, or a combination thereof. In some embodiments the endoglycosidase is Endo SH.

In some embodiments, step (b) of the process (the formation of the modified B7-H4 antibody) wherein the intermediate antibody comprises a monoclonal antibody (mAb) and a terminal GlcNAc moiety (which optionally comprises a fucose (e.g., u₃ is 0 or 1)) on each heavy chain of the monoclonal antibody (mAb).

In some embodiments, the compound of P″—S″—A″ is GalNAz-UDP (e.g., 4-AzGalNAc-UDP). In some embodiments, the terminal-GlcNAc moiety is *-GlcNAc-GalNAz or *-GlcNAc(Fuc)-GalNAz, wherein * denotes the attachment to the rest of the modified B7-H4 antibody.

In some embodiments, the steps of the deglycosylation/trimming step and the formation of the modified B7-H4 antibody are conducted sequentially.

In some embodiments, the steps of the deglycosylation/trimming step and the formation of the modified B7-H4 antibody are conducted simultaneously.

In some embodiments, the process for the preparation of a modified B7-H4 antibody is performed in a suitable buffer solution, e.g., buffered saline (e.g. phosphate-buffered saline, Tris-buffered saline), citrate, HEPES, Tris and glycine. In some embodiments, the buffer solution is phosphate-buffered saline (PBS) or Tris buffered saline. In some embodiments, the buffer solution is phosphate-buffered saline (PBS).

In some embodiments, the process is performed at a temperature ranging from about 4 to about 50° C. In some embodiments, the process is performed at a temperature ranging from about to about 45° C. In some embodiments, the process is performed at a temperature ranging from about 20 to about 40° C. In some embodiments, the process is performed at a temperature ranging from about 30 to about 37° C. In some embodiments, the process is performed at a temperature of about 30° C. In some embodiments, the process is performed at a temperature of 30° C.

In some embodiments, the process is performed at a pH value ranging from about 5 to about 9 (e.g., from about 5.5 to about 8.5, from about 6 to about 8, or from about 7 to about 8). In some embodiments, the process is performed at a pH value of about 7.4.

In some embodiments, the process for the preparation of a modified B7-H4 antibody comprises:
(a) contacting a glycoprotein (e.g., a B7-H4 antibody glycan) comprising a B7-H4 antibody and core-GlcNAc moiety connected to site N297 of the antibody (according to EU numbering), with endoglycosidase Endo SH, thereby forming an intermediate antibody comprising a terminal GlcNAc moiety; and
(b) contacting the intermediate antibody with 4-AzGalNAc-UDP in the presence of a β-(1,4)-GalNAcT enzyme, thereby forming the modified B7-H4 antibody comprising the modified-GlcNAc moiety;
wherein steps (a) and (b) are conducted concurrently.

In some embodiments, the endoglycosidase is Endo SH, a fusion between the two endoglycosidases, Endo S and Endo H, linked by a Gly-rich spacer comprising an internal 6xHis tag resulting in an overall molecular weight of 139 kDa.

In some embodiments, the β-(1,4)-GalNAcT enzyme comprises an N-terminal 6xHis tag and has an overall molecular weight of 45.7 kDa. In some embodiments, the β-(1,4)-GalNAcT enzyme containing an N-terminal 6xHis tag is derived from Trichopulsia ni.

In some embodiments, the process is conducted in PBS buffer at pH value of about 7.4 and at a temperature of about 30° C.

### B7-H4-Targeted Antibody-Drug Conjugates

The invention pertains to therapies involving immunoconjugates comprising an antibody conjugated to a cytotoxic agent such as a toxin (e.g., an enzymatically active toxin of bacterial, fungal, plant, or animal origin, or fragments thereof), via site specific conjugation.

The conjugate described herein is a B7-H4-targeted antibody-drug conjugate comprising a B7-H4 modified antibody and two molecules of a fully synthetic macromolecular linker-payload bearing three copies of the microtubule inhibitor auristatin F-hydroxypropyl amide (AF-HPA). The fully synthetic macromolecular linker-payload is conjugated to the modified B7-H4 antibody in a site-specific manner enabled by glycan remodeling of the antibody at the asparagine group at position 297 of the antibody according to EU numbering.

The average drug-to-antibody ratio (DAR) of the B7-H4-targeted antibody-drug conjugate is about 6. In some embodiments, the DAR of B7-H4-targeted antibody-drug conjugate is about 2 to about 8. In some embodiments, the DAR of B7-H4-targeted antibody-drug conjugate is about 5 to about 7. In some embodiments, the DAR of B7-H4-targeted antibody-drug conjugate is about 5.5, about 5.6, about 5.7, about 5.8, about 5.9, about 6.0, about 6.1, about 6.2 about 6.3, about 6.4, or about 6.5, or any number in between. In some embodiments, the B7-H4 antibody-drug conjugate is a conjugate of Formula (I):

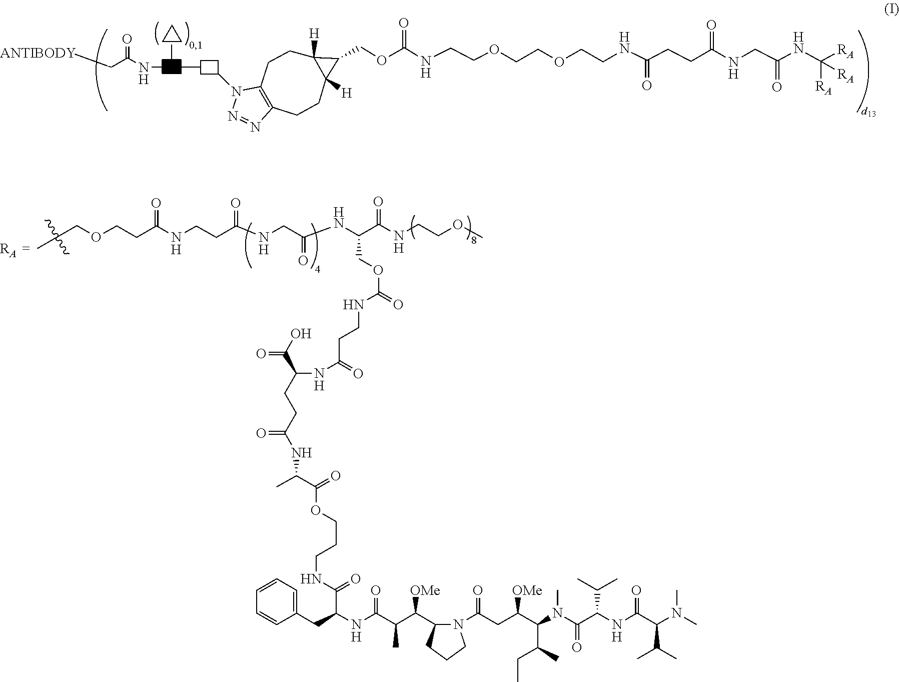

wherein
i. d₁₃ is about 2;
ii. the ANTIBODY binds B7-H4 and comprises a variable heavy chain complementarity determining region 1 (CDRH1) comprising the amino acid sequence GFIVSRNY (SEQ ID NO: 2), a variable heavy chain complementarity determining region 2 (CDRH2) comprising the amino acid sequence IYGSGRT (SEQ ID NO: 3), a variable heavy chain complementarity determining region 3 (CDRH3) comprising the amino acid sequence ARDADYGLDV (SEQ ID NO: 4), a variable light chain complementarity determining region 1 (CDRL1) comprising the amino acid sequence QSVSSSY (SEQ ID NO: 5), a variable light chain complementarity determining region 2 (CDRL2) comprising the amino acid sequence GAS (SEQ ID NO: 6), and a variable light chain complementarity determining region 3 (CDRL3) comprising the amino acid sequence QQYGSSPLYT (SEQ ID NO: 7);
iii. the drug is attached to the antibody at position 297 when numbered in accordance with EU numbering via a linker moiety;
iv. ▪ is GlcNAc; Δ is Fuc; and □ is GalNAc.

In some embodiments, d₁₃ can be from about 1 to about 3. In some embodiments, d₁₃ can be about 1.5, about 1.6, about 1.7, about 1.8, about 1.9, about 2.0, about 2.1, about 2.2, about 2.3, about 2.4, or about 2.5.

GlcNAc refers to N-acetylglucosamine (i.e., β-D-(Acetylamino)-2-deoxy-glucopyranose or N-Acetyl-D-glucosamine).

Fuc refers to fucose (i.e., (2S,3R,4R,5S)-6-Methyltetrahydro-2H-pyran-2,3,4,5-tetraol or 6-Deoxy-l-galactose).

GalNAc refers to N-acetylgalactosamine (i.e., 2-(Acetylamino)-2-deoxy-D-galactose, 2-Acetamido-2-deoxy-D-galactose, N-Acetylchondrosamine, 2-Acetamido-2-deoxy-D-galactopyranose, or N-Acetyl-D-galactosamine).

In some embodiments, the GlcNAc is bonded to the conjugate through a reactive moiety of GlcNAc.

In some embodiments, the Fuc is bonded to the conjugate through a reactive moiety of Fuc.

In some embodiments, the GalNAc is bonded to the conjugate through a reactive moiety of GalNAc.

In some embodiments, the drug is attached to the antibody at position N180 of SEQ NO: 11, a position corresponding to N297 (as numbered by EU numbering).

In some embodiments, the drug is attached to the antibody at position N296 of SEQ NO: 13, a position corresponding to N297 (as numbered by EU numbering).

In some embodiments, the process for the preparation of a modified B7-H4 antibody comprises:
(a) contacting a glycoprotein (e.g., a B7-H4 antibody glycan) comprising a B7-H4 antibody and core-GlcNAc moiety connected to site N297 (according to EU numbering) of the antibody, with endoglycosidase Endo SH, thereby forming an intermediate antibody comprising a terminal GlcNAc moiety; and
(b) contacting the intermediate antibody with 4-AzGalNAc-UDP in the presence of a β-(1,4)-GalNAcT enzyme, thereby forming the modified B7-H4 antibody comprising the modified-GlcNAc moiety;
wherein steps (a) and (b) are conducted concurrently.

In some embodiments, the endoglycosidase is Endo SH, a fusion between the two endoglycosidases, Endo S and Endo H, linked by a Gly-rich spacer comprising an internal 6xHis tag resulting in an overall molecular weight of 139 kDa.

In some embodiments, the β-(1,4)-GalNAcT enzyme comprises an N-terminal 6xHis tag and has an overall molecular weight of 45.7 kDa. In some embodiments, the β-(1,4)-GalNAcT enzyme containing an N-terminal 6xHis tag is derived from Trichopulsia ni.

In some embodiments, the process is conducted in PBS buffer at pH value of about 7.4 and at a temperature of about 30° C.

The B7-H4 antibody-drug conjugate, (e.g., XMT-1660) suitable for use in the methods disclosed herein can be generated and purified by well-known techniques e.g., WO 2018098269 and U.S. Ser. No. 17/568,378, each of which is incorporated herein in its entirety by reference.

In some embodiments, the B7-H4 antibody-drug conjugate of Formula (I) is of Formula (I-a):

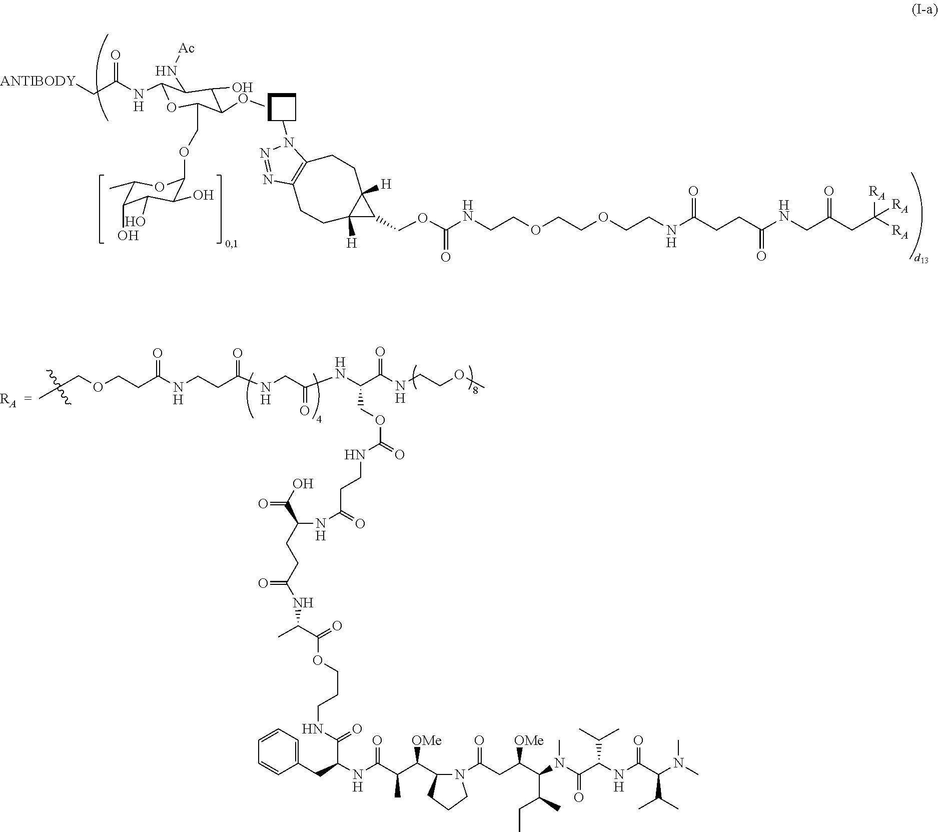

wherein is GalNAc; and
d₁₃ is about 2.

In some embodiments, the B7-H4 antibody-drug conjugate of Formula (I) is of Formula (I-b):

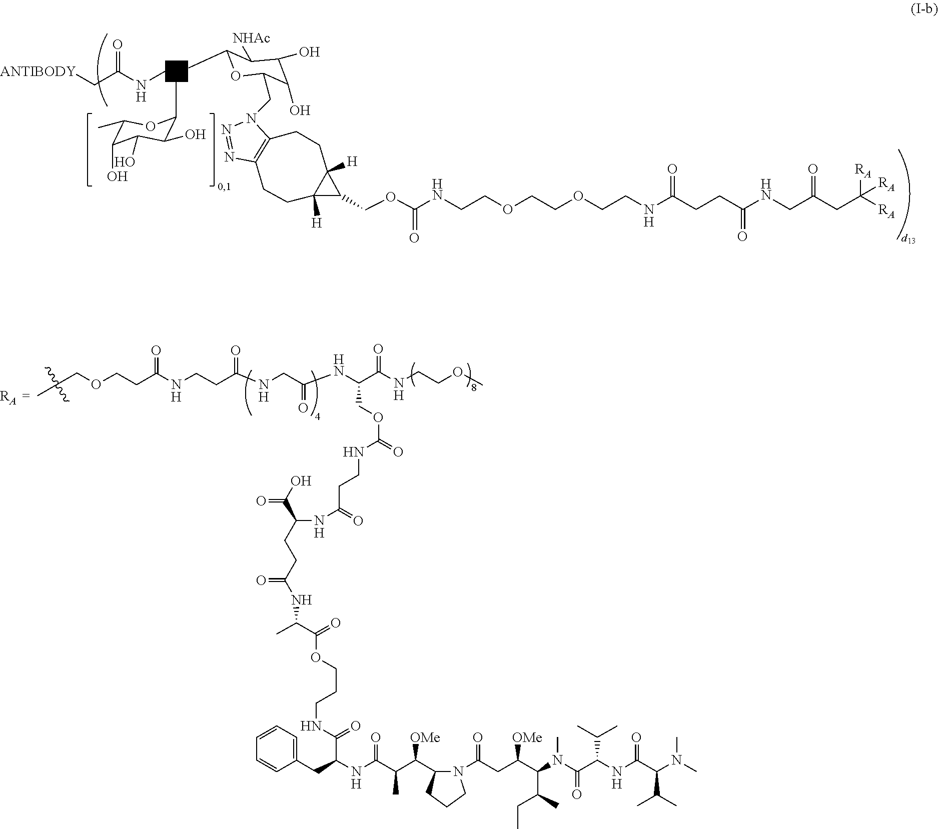

wherein ▪ is GlcNAc; and
d₁₃ is about 2.

In some embodiments, the B7-H4 antibody-drug conjugate of Formula (I) is of Formula (I-c):

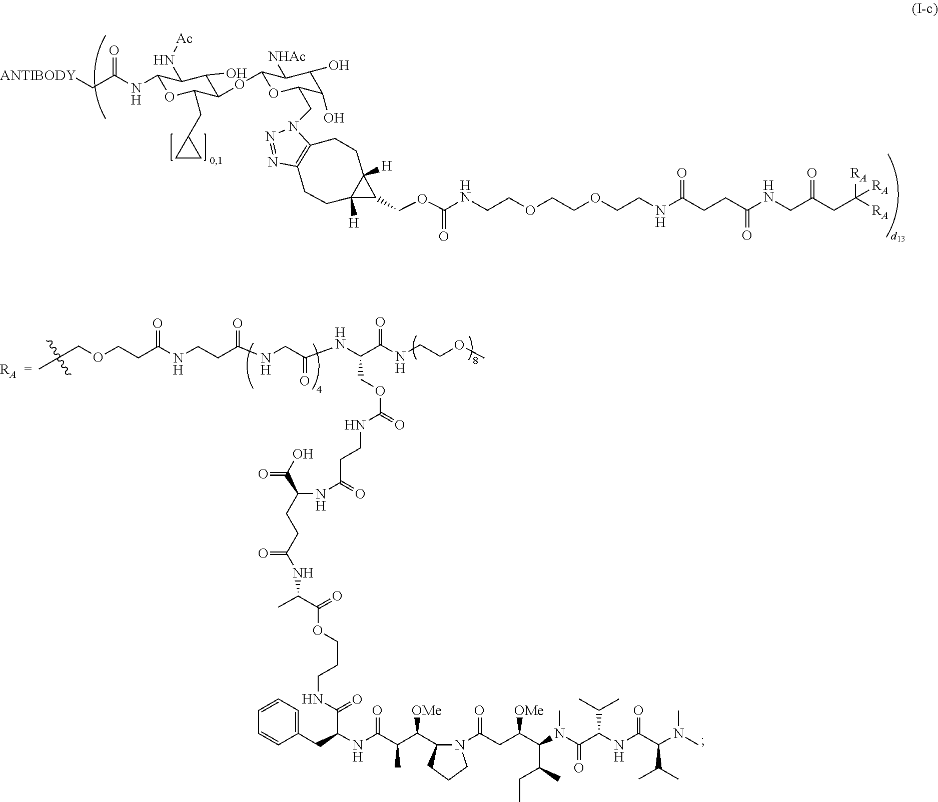

wherein Δ is Fuc; and
d₁₃ is about 2.

In some embodiments, the B7-H4 antibody-drug conjugate is a conjugate of Formula (IA):

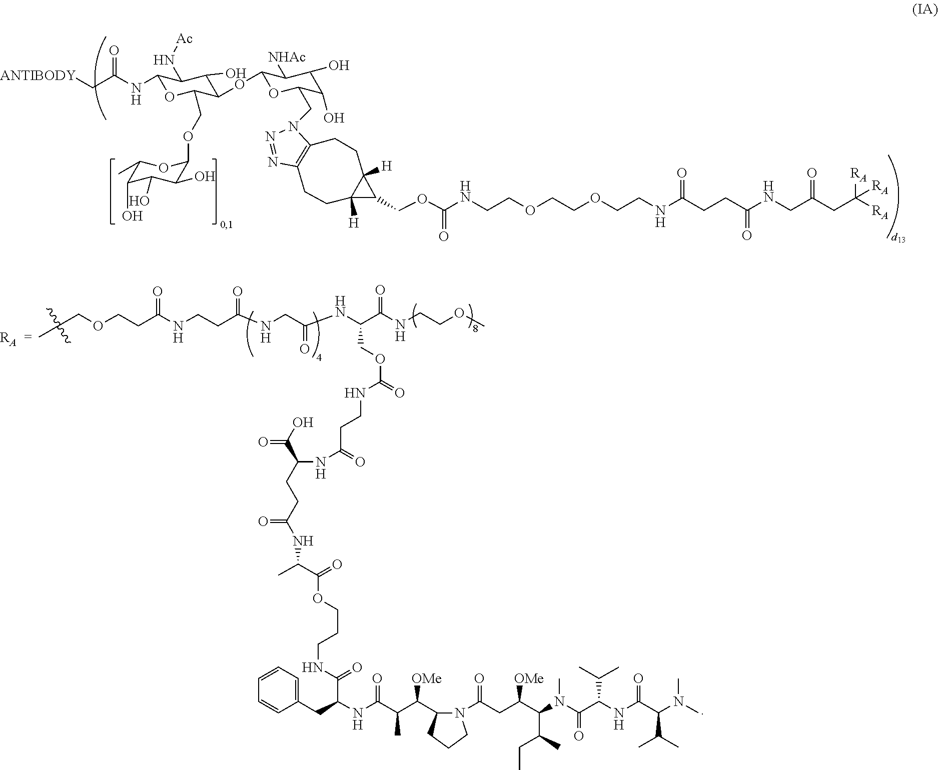

### Dosage and Administration

The cancer therapy provided herein, containing a B7-H4-targeted antibody-drug conjugate, is administered in an amount sufficient to exert a therapeutically useful effect. Typically, the active agents are administered in an amount that does not result in undesirable side effects of the patient being treated, or that minimizes or reduces the observed side effects. B7-H4-expressing cancers include for example, breast cancer, endometrial cancer, ovarian cancer, non-small cell lung cancer (e.g., squamous cell carcinoma), pancreatic cancer, thyroid cancer, kidney cancer (e.g., renal cell carcinoma), bladder cancer (e.g., urothelial cell carcinoma), colon cancer, head and neck cancer, small cell lung cancer, gastric cancer, melanoma, bile duct carcinoma, uterine cancer, and cholangial carcinoma.

It is understood that the precise dosage and duration of treatment is a function of the tissue or tumor being treated and may be determined empirically using known testing protocols or by extrapolation from in vivo or in vitro test data and/or can be determined from known dosing regimens of the particular agent. It is to be noted that concentrations and dosage values may also vary with the age of the individual treated, the weight of the individual, the route of administration and/or the extent or severity of the disease and other factors that are within the level of a skilled medical practitioner to consider. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects). It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof. For example, the B7-H4-targeted antibody-drug conjugate, is administered in a therapeutically effective amount to decrease the tumor volume.

Patients with multiple solid tumors including breast, ovarian, and endometrial cancers are administered a B7-H4-targeted antibody-drug conjugate (e.g. XMT-1660) in an amount sufficient to exert a therapeutically useful effect.

In some embodiments, the tumor or cancer is positive for B7-H4 expression.

In some embodiments, the subject has triple negative breast cancer (TNBC). In some embodiments the subject has EIR+/HER2- breast cancer. In some embodiments the subject has endometrial cancer. In other embodiments, the subject has ovarian cancer, fallopian tube cancer, or primary peritoneal cancer.

In some embodiments, the subject that has unresectable or recurrent/metastatic TNBC after standard chemotherapy. In some embodiments, the subject that has TNBC has received at least 2 lines of systemic therapy in locally advanced or metastatic breast cancer setting. In some embodiments, the patient has histologically or cytologically proven diagnosis of breast cancer with evidence of metastatic or locally advanced disease. In some embodiments, the patient has estrogen receptor (ER)-negative, progesterone receptor (PR)-negative and HER2-negative tumor based on local testing on the most recent tumor biopsy. In some embodiments, the patient has received 1-3 prior lines of chemotherapy for metastatic setting. In some embodiments, the patient with BRCA mutation has received prior treatment with a PARP inhibitor.

In some embodiments, the subject has HR+/HER2− breast cancer post-CDK4/6 inhibitor and endocrine-based therapy. In some embodiments, the subject that has HR+/HER2− breast cancer has received at least one line of systemic therapy which must have included CDK 4/6 inhibitor and endocrine therapy (ET), in an advanced or metastatic breast cancer setting. In some embodiments the subject that has HR+/HER2− breast cancer has histologically or cytologically proven diagnosis of breast cancer with evidence of metastatic or locally advanced disease. In some embodiments, the subject that has ER-positive and/or PR-positive tumor and HER2-negative tumor based on local testing on the most recent tumor biopsy.

In some embodiments, the subject that has endometrial cancer has received at least 1 line of systemic therapy including platinum-based chemotherapy for advanced or metastatic disease. In some embodiments, the subject has histologically or cytologically proven diagnosis of endometrial carcinoma, with evidence of metastatic or locally advanced disease who have no satisfactory treatment options and for whom experimental therapy is appropriate. In some embodiments, the subject has received at least 1 prior line of platinum-doublet chemotherapy and no more than 3 prior lines of systemic therapy for recurrent or metastatic cancer, not including hormonal therapy. In some embodiments, the subject that has endometrial cancer must not have endometrial sarcoma or carcinosarcoma.

In some embodiments, the subject that has ovarian cancer, fallopian tube cancer, or primary peritoneal cancer has received at least 2 lines of systemic therapy for advanced or metastatic disease, which should include platinum-based chemotherapy. In some embodiments, the subject has a histological diagnosis of high grade serous ovarian cancer, which includes fallopian tube cancer, or primary peritoneal cancer, that is metastatic or recurrent. In some embodiments, the subject must have platinum-resistant recurrent disease, defined as completing 4 or more cycles of platinum-based therapy and radiographically progressing within 6 months of last platinum-based therapy. In some embodiments, the subject who has received 1 prior line of chemotherapy, must have achieved a complete or partial response to that therapy and their disease progressing between 3 and 6 months after the last dose of platinum in the first-line setting. In some embodiments, the subject who has received 2 to 4 prior lines of chemotherapy, disease progressing within less than or equal to 6 months after the last dose of platinum in the most recent chemotherapy. In some embodiments, the subject who has received 1 to 4 prior lines of systemic therapy for ovarian cancer including at least 1 prior line of a platinum-containing regimen.

In some embodiments, the subject having TNBC, Hit+/HER2− breast cancer, endometrial cancer, ovarian cancer, fallopian tube cancer, or primary peritoneal cancer is administered by infusion, a B7-H4-targeted antibody-drug conjugate (e.g. XMT-1660) at a dosage amount according to body surface area (BSA). The BSA adjusted dose will be calculated when possible, using the Mosteller Formula. The starting dose will be calculated based on height and weight collected within 14 days of the first dose (can be collected the day of first dose). Dose calculations in subsequent cycles will be made for weight changes ≥5% from the most recent dose calculation. Dose adjustments based on subsequent weight measurements will be made. Additional weight measurements will be obtained and BSA confirmed or altered prior to dosing at Cycle 2 and every 2 cycles thereafter.

In some embodiments, administration of a B7-H4-targeted antibody-drug conjugate (e.g. XMT-1660) is via infusion. Methods of infusion can comprise any method of infusing therapeutic agents to a subject known in the art. In some embodiments the infusion is an intravenous (IV) infusion.

In some embodiments, infusions of a B7-H4-targeted antibody-drug conjugate (e.g., XMT-1660) occur over a duration of at least 1 minute, at least 5 minutes, at least 10 minutes, at least 15 minutes, at least 20 minutes, at least 25 minutes, at least 30 minutes, at least 35 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, or any number of minutes therebetween. In some embodiments, the duration of infusion can be varied from the first infusion to the second and subsequent infusions.

In some embodiments, the initial dose of a B7-H4-targeted antibody-drug conjugate (e.g., XMT-1660)for each subject will be administered over 90±10 minutes. If no infusion related reactions occur, all subsequent doses can be administered over 60±10 minutes.

In some embodiments, the subject having TNBC, HR+/HER2− breast cancer, endometrial cancer, ovarian cancer, fallopian tube cancer, or primary peritoneal cancer is administered by infusion a B7-H4-targeted antibody-drug conjugate (e.g., XMT-1660) at a dosage amount that is between about 5 mg/m² to about 100 mg/m².

In some embodiments, the dosage of the B7-H4-targeted antibody-drug conjugate is between about 5 mg/m² to about 50 mg/m² or about 50 mg/m² to about 100 mg/m².

In some embodiments, the dose is between about 5 mg/m² to about 10 mg/m², about 10 mg/m² to about 15 mg/m², about 15 mg/m² to about 20 mg/m², about 20 mg/m² to about 25 mg/m², about 25 mg/m² to about 30 mg/m², about 30 mg/m² to about 35 mg/m², about 35 mg/m² to about 40 mg/m², about 40 mg/m² to about 45 mg/m², about 45 mg/m² to about 50 mg/m², about 50 mg/m² to about 55 mg/m², about 55 mg/m² to about 60 mg/m², about 60 mg/m² to about 65 mg/m², about 65 mg/m² to about 70 mg/m², about 75 mg/m² to about 80 mg/m², about 85 mg/m² to about 90 mg/m², about 90 mg/m² to about 95 mg/m², or about 95 mg/m² to about 100 mg/m².

In some embodiments, the dosage of the B7-H4-targeted antibody-drug conjugate is about 6.0 mg/m² to about 8.0 mg/m². In some embodiments, the dosage of the B7-H4-targeted antibody-drug conjugate is about 6.7 mg/m² to about 7.7 mg/m². In some aspects, the dosage is about 6.0 mg/m², about 6.1 mg/m², about 6.2 mg/m², about 6.3 mg/m², about 6.4 mg/m², about 6.5 mg/m², about 6.6 mg/m², about 6.7 mg/m², about 6.8 mg/m², about 6.9 mg/m², about 7.0 mg/m², about 7.1 mg/m², about 7.2 mg/m², about 7.3 mg/m², about 7.4 mg/m², about 7.5 mg/m², about 7.6 mg/m², about 7.7 mg/m², about 7.8 mg/m², about 7.9 mg/m², or about 8.0 mg/m². In some embodiments, the dosage of the B7-H4-targeted antibody-drug conjugate is about 7.2 mg/m².

In some embodiments, the dosage of the B7-H4-targeted antibody-drug conjugate is about 12.0 mg/m² to about 15.0 mg/m². In some embodiments, the dosage of the B7-H4-targeted antibody-drug conjugate is about 13.9 mg/m² to about 14.9 mg/m². In some aspects, the dosage is about 13.0 mg/m², about 13.1 mg/m², about 13.2 mg/m², about 13.3 mg/m², about 13.4 mg/m², about 13.5 mg/m², about 13.6 mg/m², about 13.7 mg/m², about 13.8 mg/m², about 13.9 mg/m², about 14.0 mg/m², about 14.1 mg/m², about 14.2 mg/m², about 14.3 mg/m², about 14.4 mg/m², about 14.5 mg/m², about 14.6 mg/m², about 14.7 mg/m², about 14.8 mg/m², about 14.9 mg/m², or about 15.0 mg/m². In some embodiments, the dosage of the B7-H4-targeted antibody-drug conjugate is about 14.4 mg/m².

In some embodiments, the dosage of the B7-H4-targeted antibody-drug conjugate is about 20.0 mg/m² to about 23.0 mg/m². In some embodiments, the dosage of the B7-H4-targeted antibody-drug conjugate is about 21.1 mg/m² to about 22.1 mg/m². In some aspects, the dosage is about 20.0 mg/m², about 20.1 mg/m², about 20.2 mg/m², about 20.3 mg/m², about 20.4 mg/m², about 20.5 mg/m², about 20.6 mg/m², about 20.7 mg/m², about 20.8 mg/m², about 20.9 mg/m², about 21.0 mg/m², about 21.1 mg/m², about 21.2 mg/m², about 21.3 mg/m², about 21.4 mg/m², about 21.5 mg/m², about 21.6 mg/m², about 21.7 mg/m², about 21.8 mg/m², about 21.9 mg/m², about 22.0 mg/m², about 22.1 mg/m², about 22.2 mg/m², about 22.3 mg/m², about 22.4 mg/m², about 22.5 mg/m², about 22.6 mg/m², about 22.7 mg/m², about 22.8 mg/m², about 22.9 mg/m², or about 23.0 mg/m². In some embodiments, the dosage of the B7-H4-targeted antibody-drug conjugate is about 21.6 mg/m².

In some embodiments, the dosage of the B7-H4-targeted antibody-drug conjugate is about 27.0 mg/m² to about 30.0 mg/m². In some embodiments, the dosage of the B7-H4-targeted antibody-drug conjugate is about 28.2 mg/m² to about 29.2 mg/m². In some aspects, the dosage is about 27.0 mg/m², about 27.1 mg/m², about 27.2 mg/m², about 27.3 mg/m², about 27.4 mg/m², about 27.5 mg/m², about 27.6 mg/m², about 27.7 mg/m², about 27.8 mg/m², about 27.9 mg/m², about 28.0 mg/m², about 28.1 mg/m², about 28.2 mg/m², about 28.3 mg/m², about 28.4 mg/m², about 28.5 mg/m², about 28.6 mg/m², about 28.7 mg/m², about 28.8 mg/m², about 28.9 mg/m², about 29.0 mg/m², about 29.1 mg/m², about 29.2 mg/m², about 29.3 mg/m², about 29.4 mg/m², about 29.5 mg/m², about 29.6 mg/m², about 29.7 mg/m², about 29.8 mg/m², about 29.9 mg/m², or about 30.0 mg/m². In some embodiments, the dosage of the B7-H4-targeted antibody-drug conjugate is about 28.7 mg/m².

In some embodiments, the dosage of the B7-H4-targeted antibody-drug conjugate is about 36.0 mg/m² to about 40.0 mg/m². In some embodiments, the dosage of the B7-H4-targeted antibody-drug conjugate is about 37.6 mg/m² to about 38.6 mg/m². In some aspects, the dosage is about 36.0 mg/m², about 36.1 mg/m², about 36.2 mg/m², about 36.3 mg/m², about 36.4 mg/m², about 36.5 mg/m², about 36.6 mg/m², about 36.7 mg/m², about 36.8 mg/m², about 36.9 mg/m², about 37.0 mg/m², about 37.1 mg/m², about 37.2 mg/m², about 37.3 mg/m², about 37.4 mg/m², about 37.5 mg/m², about 37.6 mg/m², about 37.7 mg/m², about 37.8 mg/m², about 37.9 mg/m², about 38.0 mg/m², about 38.1 mg/m², about 38.2 mg/m², about 38.3 mg/m², about 38.4 mg/m², about 38.5 mg/m², about 38.6 mg/m², about 38.7 mg/m², about 38.8 mg/m², about 38.9 mg/m², about 39.0 mg/m², about 39.1 mg/m², about 39.2 mg/m², about 39.3 mg/m², about 39.4 mg/m², about 39.5 mg/m², about 39.6 mg/m², about 39.7 mg/m², about 39.8 mg/m², about 39.9 mg/m², about 40.0 mg/m². In some embodiments, the dosage of the B7-H4-targeted antibody-drug conjugate is about 38.1 mg/m².

In some embodiments, the dosage of the B7-H4-targeted antibody-drug conjugate is about 49.0 mg/m² to about 52.0 mg/m². In some embodiments, the dosage of the B7-H4-targeted antibody-drug conjugate is about 50.2 mg/m² to about 51.2 mg/m². In some aspects, the dosage is about 49.0 mg/m², about 49.1 mg/m², about 49.2 mg/m², about 49.3 mg/m², about 49.4 mg/m², about 49.5 mg/m², about 49.6 mg/m², about 49.7 mg/m², about 49.8 mg/m², about 49.9 mg/m², about 50.0 mg/m², about 50.1 mg/m², about 50.2 mg/m², about 50.3 mg/m², about 50.4 mg/m², about 50.5 mg/m², about 50.6 mg/m², about 50.7 mg/m², about 50.8 mg/m², about 50.9 mg/m², about 51.0 mg/m², about 51.1 mg/m², about 51.2 mg/m², about 51.3 mg/m², about 51.4 mg/m², about 51.5 mg/m², about 51.6 mg/m², about 51.7 mg/m², about 51.8 mg/m², about 51.9 mg/m², or about 52.0 mg/m². In some embodiments, the dosage of the B7-H4-targeted antibody-drug conjugate is about 50.7 mg/m².

In some embodiments, the dosage of the B7-H4-targeted antibody-drug conjugate is about 66.0 mg/m² to about 69.0 mg/m². In some embodiments, the dosage of the B7-H4-targeted antibody-drug conjugate is about 66.9 mg/m² to about 67.9 mg/m². In some aspects, the dosage is about 66.0 mg/m², about 66.1 mg/m², about 66.2 mg/m², about 66.3 mg/m², about 66.4 mg/m², about 66.5 mg/m², about 66.6 mg/m², about 66.7 mg/m², about 66.8 mg/m², about 66.9 mg/m², about 67.0 mg/m², about 67.1 mg/m², about 67.2 mg/m², about 67.3 mg/m², about 67.4 mg/m², about 67.5 mg/m², about 67.6 mg/m², about 67.7 mg/m², about 67.8 mg/m², about 67.9 mg/m², about 68.0 mg/m², about 68.1 mg/m², about 68.2 mg/m², about 68.3 mg/m², about 68.4 mg/m², about 68.5 mg/m², about 68.6 mg/m², about 68.7 mg/m², about 68.8 mg/m², about 68.9 mg/m², or about 69.0 mg/m². In some embodiments, the dosage of the B7-H4-targeted antibody-drug conjugate is about 67.4 mg/m².

In some embodiments, the dosage of the B7-H4-targeted antibody-drug conjugate is about 86.0 mg/m² to about 89.0 mg/m². In some embodiments, the dosage of the B7-H4-targeted antibody-drug conjugate is about 87.1 mg/m² to about 88.1 mg/m². In some aspects, the dosage is about 86.0 mg/m², about 86.1 mg/m², about 86.2 mg/m², about 86.3 mg/m², about 86.4 mg/m², about 86.5 mg/m², about 86.6 mg/m², about 86.7 mg/m², about 86.8 mg/m², about 86.9 mg/m², about 87.0 mg/m², about 87.1 mg/m², about 87.2 mg/m², about 87.3 mg/m², about 87.4 mg/m², about 87.5 mg/m², about 87.6 mg/m², about 87.7 mg/m², about 87.8 mg/m², about 87.9 mg/m², about 88.0 mg/m², about 88.1 mg/m², about 88.2 mg/m², about 88.3 mg/m², about 8.4 mg/m², about 88.5 mg/m², about 88.6 mg/m², about 88.7 mg/m², about 88.8 mg/m², about 88.9 mg/m², or about 89.0 mg/m². In some embodiments, the dosage of the B7-H4-targeted antibody-drug conjugate is about 87.6 mg/m². In some embodiments, the dosage of the B7-H4-targeted antibody-drug conjugate is about 1.0 mg/m², about 2.0 mg/m², about 3.0 mg/m², about 4.0 mg/m², about 5.0 mg/m², about 6.0 mg/m², about 7.0 mg/m², about 8.0 mg/m², about 9.0 mg/m², about 10.0 mg/m², about 11.0 mg/m², about 12.0 mg/m², about 13.0 mg/m², about 14.0 mg/m², about 15.0 mg/m², about 16.0 mg/m², about 17.0 mg/m², about 18.0 mg/m², about 19.0 mg/m², about 20.0 mg/m², about 21.0 mg/m², about 22.0 mg/m², about 23.0 mg/m², about 24.0 mg/m², about 25.0 mg/m², about 26.0 mg/m², about 27.0 mg/m², about 28.0 mg/m², about 29.0 mg/m², about 30.0 mg/m², about 31.0 mg/m², about 32.0 mg/m², about 33.0 mg/m², about 34.0 mg/m², about 35.0 mg/m², about 36.0 mg/m², about 37.0 mg/m², about 38.0 mg/m², about 39.0 mg/m², about 40.0 mg/m², about 41.0 mg/m², about 42.0 mg/m², about 43.0 mg/m², about 44.0 mg/m², about 45.0 mg/m², about 46.0 mg/m², about 47.0 mg/m², about 48.0 mg/m², about 49.0 mg/m², about 50.0 mg/m², about 51.0 mg/m², about 52.0 mg/m², about 53.0 mg/m², about 54.0 mg/m², about 55.0 mg/m², about 56.0 mg/m², about 57.0 mg/m², about 58.0 mg/m², about 59.0 mg/m², about 60.0 mg/m², about 61.0 mg/m², about 62.0 mg/m², about 63.0 mg/m², about 64.0 mg/m², about 65.0 mg/m², about 66.0 mg/m², about 67.0 mg/m², about 68.0 mg/m², about 69.0 mg/m², about 70.0 mg/m², about 71.0 mg/m², about 72.0 mg/m², about 73.0 mg/m², about 74.0 mg/m², about 75.0 mg/m², about 76.0 mg/m², about 77.0 mg/m², about 78.0 mg/m², about 79.0 mg/m², about 80.0 mg/m², about 81.0 mg/m², about 82.0 mg/m², about 83.0 mg/m², about 84.0 mg/m², about 85.0 mg/m², about 86.0 mg/m², about 87.0 mg/m², about 88.0 mg/m², about 89.0 mg/m², about 90.0 mg/m², about 91.0 mg/m², about 92.0 mg/m², about 93.0 mg/m², about 94.0 mg/m², about 95.0 mg/m², about 96.0 mg/m², about 97.0 mg/m², about 98.0 mg/m², about 99.0 mg/m², or about 100.0 mg/m².

In some embodiments, the subject having TNBC, HR+/HER2− breast cancer, endometrial cancer, ovarian cancer, fallopian tube cancer, or primary peritoneal cancer is administered by infusion a B7-H4-targeted antibody-drug conjugate (e.g. XMT-1660) at a dosage amount that is between about 7.2 mg/m² to about 87.6 mg/m². In some embodiments, the dosage of a B7-H4-targeted antibody-drug conjugate (e.g. XMT-1660) is about 7.2 mg/m². In some embodiments, the dosage of a B7-H4-targeted antibody-drug conjugate (e.g. XMT-1660) is about 14.4 mg/m². In some embodiments, the dosage of a B7-H4-targeted antibody-drug conjugate (e.g. XMT-1660) is about 21.6 mg/m². In other embodiments, the dosage of a B7-H4-targeted antibody-drug conjugate (e.g. XMT-1660) is about 28.7 mg/m². In some embodiments, the dosage of a B7-H4-targeted antibody-drug conjugate (e.g. XMT-1660) is about 38.1 mg/m². In other embodiments, the dosage of a B7-H4-targeted antibody-drug conjugate (e.g. XMT-1660) is about 50.7 mg/m². In some embodiments, the dosage of a B7-H4-targeted antibody-drug conjugate (e.g. XMT-1660) is about 67.4 mg/m². In some embodiments, the dosage of a B7-H4-targeted antibody-drug conjugate (e.g. XMT-1660) is about 87.6 mg/m².

In some embodiments, the dosage amounts are administered intravenously once every week (i.e., 7-day cycle), once every two weeks (i.e., 14-day cycle), three weeks (i.e., 21-day cycle) or once every four weeks (i.e., 28-day cycle).

In some embodiments, dosage amounts of the B7-H4-targeted antibody-drug conjugae are administered every one week (i.e. 7 day cycle), every two weeks (i.e. 14 day cycle), every three weeks (i.e. 21 day cycle), every four weeks (i.e. 28 day cycle), every five weeks (i.e. 35 day cycle), every six weeks (i.e. 42 day cycle), every seven weeks (i.e. 49 day cycle), or every eight weeks (i.e. 56 day cycle).

In some embodiments, a B7-H4-targeted antibody-drug conjugate (e.g. XMT-1660) infusion is at at a dosage amount that is between about 7.2 mg/m² to about 87.6 mg/m² is administered as an infusion every one week (i.e. 7 day cycle), every two weeks (i.e. 14 day cycle), every three weeks (i.e. 21 day cycle), every four weeks (i.e. 28 day cycle), every five weeks (i.e. 35 day cycle), every six weeks (i.e. 42 day cycle), every seven weeks (i.e. 49 day cycle), or every eight weeks (i.e. 56 day cycle).

In some embodiments the subject is administered a B7-H4-targeted antibody-drug conjugate (e.g. XMT-1660) at a dosage amount that is between about 7.2 mg/m² to about 87.6 mg/m² once every 3 weeks (i.e. 21 day cyle). In some embodiments the subject is administered XMT-1660 at a dosage amount that is between about 7.2 mg/m² to about 87.6 mg/m² once every 4 weeks (i.e. 28 day cyle).

In some embodiments the subject is administered a B7-H4-targeted antibody-drug conjugate (e.g. XMT-1660) at a dosage amount that is between about 7.2 mg/m² to about 87.6 mg/m² over 90 min for the first infusion, then over 90 minutes for the subsequent infusions once every 3 weeks (i.e. 21 day cycle).

In some embodiments the subject is administered a B7-H4-targeted antibody-drug conjugate (e.g. XMT-1660) at a dosage amount that is between about 7.2 mg/m² to about 87.6 mg/m² over 90 min for the first infusion, then over 60 minutes for the subsequent infusions once every 3 weeks (i.e. 21 day cycle).

In some embodiments the subject is administered a B7-H4-targeted antibody-drug conjugate (e.g. XMT-1660) at a dosage amount that is between about 7.2 mg/m² to about 87.6 mg/m² over 90 min for the first infusion, then over 30 minutes for the subsequent infusions once every 4 weeks (i.e. 28 day cycle).

In some embodiments the subject is administered a B7-H4-targeted antibody-drug conjugate (e.g. XMT-1660) at a dosage of about 7.2 mg/m² over 90 min for the first infusion, then over 60 or 90 minutes for the subsequent infusions once every 3 weeks (i.e. 21 day cycle).

In some embodiments the subject is administered a B7-H4-targeted antibody-drug conjugate (e.g. XMT-1660) at a dosage of about 14.4 mg/m² over 90 min for the first infusion, then over 60 or 90 minutes for the subsequent infusions once every 3 weeks.

In some embodiments the subject is administered a B7-H4-targeted antibody-drug conjugate (e.g. XMT-1660) at a dosage of about 21.6 mg/m² over 90 min for the first infusion, then over 60 or 90 minutes for the subsequent infusions once every 3 weeks.

In some embodiments the subject is administered a B7-H4-targeted antibody-drug conjugate (e.g. XMT-1660) at a dosage of about 28.7 mg/m² over 90 min for the first infusion, then over 60 or 90 minutes for the subsequent infusions once every 3 weeks.

In some embodiments the subject is administered a B7-H4-targeted antibody-drug conjugate (e.g. XMT-1660) at a dosage of about 38.1 mg/m² over 90 min for the first infusion, then over 60 or 90 minutes for the subsequent infusions once every 3 weeks.

In some embodiments the subject is administered a B7-H4-targeted antibody-drug conjugate (e.g. XMT-1660) at a dosage of about 50.7 mg/m² over 90 min for the first infusion, then over 60 or 90 minutes for the subsequent infusions once every 3 weeks.

In some embodiments the subject is administered a B7-H4-targeted antibody-drug conjugate (e.g. XMT-1660) at a dosage of about 67.4 mg/m² over 90 min for the first infusion, then over 60 or 90 minutes for the subsequent infusions once every 3 weeks.

In some embodiments the subject is administered XMT-1660 at a dosage of about 87.6 mg/m² over 90 min for the first infusion, then over 60 or 90 minutes for the subsequent infusions once every 3 weeks.

In some embodiments the subject is administered a B7-H4-targeted antibody-drug conjugate (e.g. XMT-1660) at a dosage of 7.2 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 14.4 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 21.6 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 28.7 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 38.1 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 50.7 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 67.4 mg/m² over 90 min for the first infusion, then over 60 or 90 minutes for the subsequent infusions once every 4 weeks.

In some embodiments the subject is administered a B7-H4-targeted antibody-drug conjugate (e.g. XMT-1660) at a dosage of about 87.6 mg/m² over 90 min for the first infusion, then over 60 or 90 minutes for the subsequent infusions once every 4 weeks.

The frequency and timing of administration, and the dosage amounts, can be administered periodically over a cycle of administration to maintain a continuous and/or long term effect of the active agents for a desired length of time. The provided compositions of a B7-H4-targeted antibody-drug conjugate can be administered hourly, daily, weekly, monthly, yearly or once. The length of time of the cycle of administration can be empirically determined, and is dependent on the disease to be treated, the severity of the disease, the particular patient, and other considerations within the level of skill of the treating physician. The length of time of treatment with a combination therapy provided herein can be one week, two weeks, one months, several months, one year, several years or more.

For example, the frequency of administration of the B7-H4-targeted antibody-drug conjugate is once a day, every other day, twice weekly, once weekly, once every 2 weeks, once every 3 weeks or once every 4 weeks. The dosage can be divided into a plurality of cycles of administration during the course of treatment. For example, the B7-H4-targeted antibody-drug conjugate can be administered at the frequency over a period of about a month, 2 months, 3 months, 4 months, 5 months, 6 months, a year or more. The frequency of administration can be the same throughout the period of the cycle or can differ. For example, an exemplary dosage frequency is two times a week at least for a first week of a cycle of administration. After the first week, the frequency can continue at twice a week, can increase to more than twice a week, or can be reduced to no more than once a week. It is within the level of a skilled person to determine the particular dosage frequency and cycle of administration based on the particular dosage being administered, the disease or condition being treated, the severity of the disease or condition, the age of the subject and other similar factors.

If disease symptoms persist in the absence of discontinued treatment, treatment can be continued for an additional length of time. Over the course of treatment, evidence of disease and/or treatment-related toxicity or side effects can be monitored.

The cycle of administration of the B7-H4-targeted antibody-drug conjugate can be tailored to add periods of discontinued treatment in order to provide a rest period from exposure to the agents. The length of time for the discontinuation of treatment can be for a predetermined time or can be empirically determined depending on how the patient is responding or depending on observed side effects. For example, the treatment can be discontinued for one week, two weeks, three weeks, one month or several months. Generally, the period of discontinued treatment is built into a cycle of dosing regimen for a patient.

An exemplary dosing regimen is a treatment cycle or cycle of administration of 21 days or 28 days. Preferably, the dosing regimen is a treatment cycle or cycle of administration is 28 days. The B7-H4-targeted antibody-drug conjugate disclosed herein, is administered on day 1, followed by 20 days without dosing or is administered on day 1, followed by 27 days without dosing. It is within the level of one of skill in the art to determine the precise cycle of administration and dosing schedule.

As noted above, the cycle of administration can be for any desired length of time. Hence, the 21-day cycle or 28-day cycle of administration can be repeated for any length of time. For example, the 21-day cycle or 28-day cycle of administration can be repeated for 2 months, 3, months, 4 months, 5, months, 6 months, 7 months, 8 months, 9 months, 10 months, 11 months, 12 months, 1.5 years, 2 years, 2.5 years, 3 years or more. It is within the level of skill of the treating physician to adopt a cycle of administration and dosing regimen that meets the needs of the patient depending on personal considerations specific to the patient and disease to be treated.

In some embodiments, the subject is administered a B7-H4-targeted antibody-drug conjugate (e.g. XMT-1660) until disease progression, death, unacceptable toxicity or voluntary withdrawal—whichever comes first.

### Dose Reductions and Delays

In some cases, toxicity or adverse reactions may occur in response to the initial dose of a B7-H4-targeted antibody-drug conjugate (e.g. XMT-1660) at the doses described herein. Subsequent doses may be reduced or time between doses may be delayed or extended. In some embodiments, toxicity or adverse reactions that may result in a dose reduction or delay include, but are not limited to, increased ALT or AST levels, hepatotoxicity, interstitial lung disease (ILD), pneumonitis, hematologic toxicity, proteinuria, fatigue, nausea, other clinically significant non-laboratory toxicities, other clinically significant adverse events.

### Measurement of B7-H4 Expression

In various embodiments the invention provides a method for identifying a cancer patient amenable to B7-H-targeted therapy or monitoring the treatment regimen by measuring the status of B7-H4 expression in a tumor sample obtained from the patient.

In some embodiments, the B7-H4 diagnostic tests can be used to identify subjects for treatment with the B7-H4-targeted drug conjugate.

The sample is derived from the subject having a cancer. The sample of cancer cells is dissected from tissue removed or obtained from the subject. In some embodiments, the sample is a fresh, frozen or an archival biopsy sample.

In some embodiments, the test cell population is derived from fresh, unfrozen tissue from a biopsy sample. In other embodiments, the test cell population is derived from a primary or metastatic site. In some embodiments, the test cell population is derived from a fresh or frozen tissue from a biopsy or surgical sample or ascitic fluid or pleural fluid. In some embodiments, the test cell population is derived from a fixed tissue (e.g., formalin fixation or formalin-fixed paraffin-embedded (FFPE)) from a biopsy or surgical sample or cell block derived from a fluid specimen. The tissue sample may be frozen or fresh.

The requisite level of B7-H4 expression may be that which is identified by the any methods known in the art and more specifically by the methods described herein. For example, the level of B7-H4 expression can be measured by conducting a known immunological assay, such as an enzyme immunoassay, radioimmunoassay, competitive immunoassay, double antibody sandwich assay, fluoroimmuno assay, ELISA, Western blotting technique, agglutination assay, cytofluorometry (e.g. flow cytometry), Fluorescence in situ hybridization (FISH), colorimetric or immunohistochemical staining assay (IHC) for protein expression using an antibody that specifically recognizes B7-H4. Cell-based assays, such as, for example, flow cytometry (FC), immuno-histochemistry (IHC), RNA expression analysis or immunofluorescence (IF) are particularly desirable in determining B7-H4 expression status, since such assay formats are clinically-suitable.

Flow cytometry (FC) may be employed to determine cell surface expression of B7-H4 in a tumor sample before, during, and after treatment with a drug. For example, tumor cells may be analyzed by flow cytometry for B7-H4 expression, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with B7-H4-specific antibody, washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed B7-H4 in the tumor.

Immunohistochemical (IHC) staining may be also employed to determine the expression of B7-H4 in a tumor sample before, during, and after treatment with a drug. IHC may be carried out according to well-known techniques. See, e.g., ANTIBODIES; A LABORATORY MANUAL, Chapter 10, Harlow & Lane Eds., Cold Spring Harbor Laboratory (1988). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary polypeptide antibody and secondary antibody; and finally detecting using ABC avidin/biotin method according to manufacturer's instructions.

Immunofluorescence (IF) assays may be also employed to determine the expression of B7-H4 tumor sample before, during, and after treatment with a drug. IF may be carried out according to well-known techniques. See, e.g., J. M. Polak and S. Van Noorden (1997) INTRODUCTION TO IMMUNOCYTOCHEMISTRY, 2nd Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with the primary antibody against polypeptide followed by a secondary antibody labeled with a fluorescent dye such as Alexa 488 and analyzed with an epifluorescent microscope.

Antibodies employed in the above-described assays may be advantageously conjugated to fluorescent dyes (e.g. Alexa488, PE), or other labels, such as quantum dots, for use in multi-parametric analyses along with other signal transduction (phospho-AKT, phospho-Erk 1/2) and/or cell marker (cytokeratin) antibodies.

In one embodiment the expression of B7-H4 in a sample from a tumor is determined immunohistochemically, using a system, for example, a Leica Bond III Fully automated Stainer (BOND III) system.

Alternatively, the assay may include preparing RNA from the sample, optionally for use in PCR (polymerase chain reaction) or other analytical methodology. The PCR methodology is optionally, for example, RT-PCR (reverse transcription-PCR) or quantitative PCR, such as, for example, real-time RT-PCR, RNA seq and the like. Alternatively, the assaying may be conducted by use of an array, such as a microarray as known in the relevant field, such as, for example, nanostring technologies.

Patients are identified as being responsive to treatment, wherein the treatment is monitored or cancer is detected by detecting and/or measuring the expression level of B7-H4 in the tumor cells in a sample.

The detection/measurement of the expression level of B7-H4 is determined by calculating a B7-H4 score. The B7-H4 score is quantitative or semi quantitative. For example, detection is scored pathologically to arrive at a pathology score. It is contemplated that any scoring methods known in the art may be used in the methods of the invention. In particular, any histological scoring methods known in the art.

The methods for assessing the measurement results obtained by immunohistochemical staining assays include, for example, the H-score method, TPS (tumor proportion score) or PS2+ (percent score) score. The H-score (Am J Clin Pathol. 1988; 90 (3): 233-9), TPS score and PS2+ scores are determined by the following calculation formula. H-Score=((% at 0)×0)+((% at 1+)×1)+((% at 2+)×2)+((% at 3+)×3), TPS-score=(% at 1+)+(% at 2+)+(% at 3+); and PS2+ score=(% at 2+)+(% at 3+); where staining intensity 0 is unstained; staining intensity 1 is weak staining; staining intensity 2 is moderate staining; and staining intensity 3 is strong staining. In some embodiments the subject having a TPS of ≥75 will be considered B7-H4 positive (high) in this assay and a TPS of <75 will be considered B7-H4 negative (low) when TPS is scored as tumor cell membrane reactivity.

In assessment by the scoring method, only cancer cell portions are used. For negative or positive controls for staining intensity, formalin-fixed paraffin-embedded cell lines or xenografts (lines whose protein expression levels are known in advance) may be employed. When there are no control specimens, a plurality of specimens are assessed simultaneously to confirm the overall distribution of staining intensity of the specimens, and then staining intensity may be set.

In addition to the scoring methods mentioned above, other scoring methods known in the art, such as, for example, the Allred method (Harvey, et al. Journal of Clinical Oncology 17, No. 5 (May 1999) 1474-1474), can also be used. Cut-off points are required to be set in each method. Allred score=score of percentage of positive cells+staining intensity score.

The disclosure also provides kits and/or methods for identifying or otherwise refining, e.g., stratifying, a patient population suitable for therapeutic administration of a B7-H4-targeted antibody-drug conjugates disclosed herein by identifying the B7-H4 score of the subject prior to treatment with a B7-H4-targeted antibody-drug conjugate disclosed herein. In some embodiments, the test cell population is derived from fresh, unfrozen tissue from a biopsy sample. In some embodiments, the test cell population is derived from a primary or metastatic site. In some embodiments, the test cell population is derived from a frozen tissue from a biopsy or surgical sample or ascetic fluid or pleural fluid. In some embodiments, the test cell population is derived from a fixed tissue (e.g., formalin fixation) from a biopsy or surgical sample. The IHC test measures the amount of B7-H4 receptor protein on the surface of cells in a cancer tissue sample

### DEFINITIONS

As utilized in accordance with the present disclosure, the following terms, unless otherwise indicated, shall be understood to have the following meanings:

As used herein, the terms “anti-B7-H4 antibody”, “B7-H4 antibody” and “an antibody that binds to B7-H4” refer to an antibody that is capable of binding B7-H4 with sufficient affinity such that the antibody is useful as a diagnostic and/or therapeutic agent in targeting B7-H4.

The term “B7-H4,” as used herein, refers to any native, mature B7-H4 which results from processing of a B7-H4 precursor protein in a cell. The term includes B7-H4 from any vertebrate source, including mammals such as primates (e.g. humans and cynomolgus monkeys) and rodents (e.g., mice and rats), unless otherwise indicated. The term also includes naturally occurring variants of B7-H4, e.g., splice variants or allelic variants.

The term “B7-H4-positive cancer” refers to a cancer comprising cells that express B7-H4 on their surface. In some embodiments, expression of B7-H4 on the cell surface is determined, for example, using antibodies to B7-H4 in a method such as immunohistochemistry, FACS, etc.

Alternatively, B7-H4 mRNA expression is considered to correlate to B7-H4 expression on the cell surface and can be determined by a method selected from in situ hybridization and RT-PCR (including quantitative RT-PCR).

The term “B7-H4-positive cell” refers to a cell that expresses B7-H4 on its surface.

The term “antibody” as used herein, is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Various methods are known in the art for numbering the amino acids sequences of antibodies and identification of the complementary determining regions. For example, the Kabat numbering system (See Kabat, E. A., et al., Sequences of Protein of immunological interest, Fifth Edition, US Department of Health and Human Services, US Government Printing Office (1991)) or the IMGT numbering system (See IMGT®, the international ImMunoGeneTics information system®. Available online: http://www.imgt.org/). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (See Lefranc. M. P. et al., Biomolecules, 2014 Dec; 4(4), 1102-1139). IMGT tools and databases run against IMGT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (See Lefranc. M. P. et al., Biomolecules, 2014 Dec; 4(4), 1102-1139). The EU numbering system (See Edelman, G. M. et al., Proc. Natl. Acad. USA, 63, 78-85 (1969) can also be used. In some aspects, EU numbering can be used to number amino acid positions of an antibody In some aspects, the EU numbering system can be used to determine position of the constant region of an antibody heavy chain or light chain.

The term “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “antibody that binds to the same epitope” as a reference antibody as used herein, refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgGi, IgG2, IgG3, IgG₄, IgAi, and IgA2. The heavy chain constant domains that correspond to the different classes of immunoglobulins are called α, δ, ε, γ, and μ, respectively.

The term “monoclonal antibody” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term a “naked antibody” refers to an antibody that is not conjugated to a heterologous moiety (e.g., a cytotoxic moiety or STING agonist drug moiety). The naked antibody may be present in a pharmaceutical formulation.

The term “native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHI, CH2, and CH3). Similarly, from - to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “epitope” refers to the particular site on an antigen molecule to which an antibody binds.

The term “humanized antibody” of an antibody refers to an antibody that is derived from a non-human antibody (e.g., murine) that retains or substantially retains the antigen-binding properties of the parent antibody but is less immunogenic in humans. Humanized as used herein is intended to include deimmunized antibodies.

The term “humanized form” of an antibody, e.g., a non-human antibody, refers to an antibody that has undergone humanization.

The term “competes with” or “cross-competes with” when used herein in the context of two or more antibodies, indicates that the two or more antibodies compete for binding to B7-H4, e.g., compete for B7-H4 binding. An antibody “blocks” or “cross-blocks” one or more other antibodies from binding to B7-H4 if the antibody competes with the one or more other antibodies 25% or more, with 25%-74% representing “partial block” and 75%-400% representing “full block”. Unless otherwise defined or negated by context, the terms “competes with”, “cross-competes with”, “blocks” or “cross-blocks” when used herein is also intended to cover such pairs of antibodies.

As used herein, an antibody that “specifically binds to human B7-H4” is intended to refer to an antibody that binds to human B7-H4 with a K_{D} of 1×10⁻⁷ or less, more typically 5×10⁻⁸ M or less, more typically 3×10⁻⁸ M or less, more typically 1×10⁻⁹ M or less, even more typically 5×10⁻⁹ M or less.

The term “does not substantially bind” to a protein or cells, as used herein, means does not bind or does not bind with a high affinity to the protein or cells, i.e. binds to the protein or cells with a K_{D} of 1×10⁻⁸ M or more, more preferably 1×10⁻⁵ M or more, more preferably 1×10⁻⁴ M or more, more preferably 1×10⁻³ M or more, even more preferably 1×10⁻² M or more.

The term “sugar” refers to a monosaccharide, for example glucose (Glc), galactose (Gal), mannose (Man) and fucose (Fuc). The term “sugar derivative” refers to a derivative of a monosaccharide sugar, i.e. a monosaccharide sugar comprising substituents and/or functional groups. Examples of a sugar derivative include, but are not limited to, amino sugars and sugar acids. Examples of a sugar derivative also include compounds denoted as S′(F′)_{X1}, wherein S′ is a sugar or a sugar derivative, F′ is a functional group and xi indicates the number of functional groups.

The term “core-GlcNAc moiety”, as used herein, refers to a monosaccharide, polysaccharide, or oligosaccharide moiety comprising a GlcNAc (e.g., a core-GlcNAc) which is attached to an antibody (e.g., via the C1 position of the GlcNAc). In some embodiments, the GlcNAc is attached to the antibody via an N-glycosidic bond to the amide nitrogen atom in the side chain of an asparagine amino acid of the antibody. In some embodiments, the core-GlcNAc moiety is present at a native glycosylation site of an antibody or is introduced on a different site on the antibody. In some embodiments, the core-GlcNAc moiety is a monosaccharide (e.g., the core-GlcNAc moiety is also a terminal-GlcNAc moiety). In some embodiments, the core-GlcNAc moiety further comprises a fucose, e.g., the core-GlcNAc moiety is a disaccharide core-GlcNAc-(α1-6-Fuc) moiety (which may be referred to as GlcNAc(Fuc)). Thus, when antibody comprises a core-GlcNAc moiety, the antibody may comprise a monosaccharide or a disaccharide core-GlcNAc moiety, and the core-GlcNAc moiety may further comprise a fucose (e.g., a disaccharide core-G1cNAc(Fuc) moiety). If the core-GlcNAc moiety further comprises a fucose, the fucose may be linked α-1,6 to O-6 of the core-GlcNAc moiety. A core-GlcNAc moiety further comprising a fucose may be referred to as core-GlcNAc(Fuc).

The term “core-GlcNAc” refers to the inner GlcNAc that is a portion of a polysaccharide or oligosaccharide, wherein the polysaccharide or oligosaccharide is attached to an antibody via the inner GlcNAc.

The term “terminal-GlcNAc moiety”, as used herein, refers to a moiety comprising a GlcNAc which is attached to an antibody and has a terminal functional group being available for further modification (e.g., with a compound of P″—S″—A″). In some embodiments, the terminal-GlcNAc moiety further comprises a fucose. In some embodiments, the terminal-GlcNAc moiety is formed by reacting the core-GlcNAc moiety of a glycoprotein (e.g., an antibody glycan) with an endoglycosidase.

The term “nucleotide” is used in its normal scientific meaning and refers to a molecule that is composed of a nucleobase, a five-carbon sugar (either ribose or 2-deoxyribose), and one, two or three phosphate groups. Without the phosphate group, the nucleobase and sugar compose a nucleoside. A nucleotide can thus also be referred to as a nucleoside monophosphate, a nucleoside diphosphate or a nucleoside triphosphate. The nucleobase may be adenine, guanine, cytosine, uracil or thymine.

The term “protein” is used in its normal scientific meaning and includes polypeptides comprising about 10 or more amino acids. A protein may comprise natural or unnatural amino acids.

The term “glycoprotein” is herein used in its normal scientific meaning and refers to a protein comprising one or more monosaccharide or oligosaccharide chains (“glycans”) covalently bonded to the protein. A glycan may be attached to a hydroxyl group on the protein (O-linked-glycan), to an amide function on the protein (N-glycoprotein), or to a carbon on the protein (C-glycoprotein). A glycoprotein may comprise more than one glycan, may comprise a combination of one or more monosaccharide and one or more oligosaccharide glycans, and may comprise a combination of N-linked, O-linked and C-linked glycans. It is estimated that more than 50% of all proteins have some form of glycosylation and therefore qualify as glycoprotein.

The term “glycan” is herein used in its normal scientific meaning and refers to a monosaccharide or oligosaccharide chain that is linked to a protein. Glycan thus refers to the carbohydrate-part of a glycoprotein. The glycan is attached to a protein via the C-1 carbon of one sugar, which may be without further substitution (monosaccharide) or may be further substituted at one or more of its hydroxyl groups (oligosaccharide). A naturally occurring glycan typically comprises 1 to about 10 saccharide moieties. However, when a longer saccharide chain is linked to a protein, said saccharide chain is also considered a glycan. A glycan of a glycoprotein may be a monosaccharide. A glycan may also be an oligosaccharide. An oligosaccharide chain of a glycoprotein may be linear or branched. In an oligosaccharide, the sugar that is directly attached to the protein is called the core sugar. In an oligosaccharide, a sugar that is not directly attached to the protein and is attached to at least two other sugars is called an internal sugar. In an oligosaccharide, a sugar that is not directly attached to the protein but to a single other sugar, i.e. carrying no further sugar substituents at one or more of its other hydroxyl groups, is called the terminal sugar. For the avoidance of doubt, there may exist multiple terminal sugars in an oligosaccharide of a glycoprotein, but only one core sugar. A glycan may be an O-linked glycan, an N-linked glycan, or a C-linked glycan. In a delinked glycan, a monosaccharide or oligosaccharide glycan is bonded to a C-atom in an amino acid of the protein.

The term “glycosyltransferase” refers to a superfamily of enzymes that are involved in the synthesis of complex carbohydrates present on glycoproteins and glycolipids.

The term “N-acetylgalactosaminyl transferase” (GalNAc-T) is a N-acetyl-D-galactosamine transferase enzyme that catalyzes the addition of N-acetyl-D-galactosamine to proteins.

The term “independently”, as used herein, means that where more than one substituent is selected from a number of possible substituents, those substituents may be the same or different.

The term “pharmaceutically acceptable”, as used herein, refers to those compounds, conjugates, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, the term “preventing,” “prevent,” or “protecting against” describes reducing or eliminating the onset of the symptoms or complications of such disease, condition or disorder.

A therapeutically “effective amount” is intended to mean that amount of a conjugate that, when administered to a patient in need of such treatment, is sufficient to effective treat or prevent, as defined herein. The amount of a given conjugate that will correspond to such an amount will vary depending upon factors such as the particular conjugate (e.g., the potency (pICso), efficacy (EC₅₀), and the biological half-life of the particular conjugate), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the conjugate will vary according to the identity of the mammal in need of treatment (e.g., weight), the particular conjugate and its properties (e.g., pharmacokinetic properties), disease or disorder and its severity and the specific composition and method being used, but can nevertheless be determined by one of skill in the art.

The term “composition” refers to a product that includes the specified ingredients in therapeutically effective amounts, as well as any product that results, directly, or indirectly, from combinations of the specified ingredients in the specified amounts.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The terms “conjugate(s) of the disclosure” or “conjugate(s) of the present disclosure”, as used herein, mean a conjugate as defined herein, in any form, i.e., any tautomeric form, any isomeric form, any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi- hydrates)), and mixtures of various forms.

Accordingly, included within the present disclosure are the conjugates as disclosure herein, in any salt or non-salt form and any physical form thereof, and mixtures of various forms. While such are included within the present disclosure, it will be understood that the conjugates of the present disclosure, in any salt or non-salt form, and in any physical form thereof, may have varying levels of activity, different bioavailabilities and different handling properties for formulation purposes.

It is understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

When used herein in the context of two or more antibodies, the term “competes with” or “cross-competes with” indicates that the two or more antibodies compete for binding to B7-H4, e.g., compete for B7-H4 binding in the assay described in Examples 5 or 8. An antibody “blocks” or “cross-blocks” one or more other antibodies from binding to B7-H4 if the antibody competes with the one or more other antibodies 25% or more, with 25%-74% representing “partial block” and 75%-400% representing “full block”, preferably as determined using the assay of Examples 5 and 8. For some pairs of antibodies, competition or blocking in the assay of the Examples 5 or 8 is only observed when one antibody is coated on the plate and the other is used to compete, and not vice versa. Unless otherwise defined or negated by context, the terms “competes with”, “cross-competes with”, “blocks” or “cross-blocks” when used herein is also intended to cover such pairs of antibodies.

As used herein, “dosing regimen” or “dosage regimen” refers to the amount of agent, for example, the composition containing an B7-H4-targeted antibody-drug conjugate, administered, and the frequency of administration. The dosing regimen is a function of the disease or condition to be treated, and thus can vary.

As used herein, “frequency” of administration refers to the time between successive administrations of treatment. For example, frequency can be days, weeks or months. For example, frequency can be more than once weekly, for example, twice a week, three times a week, four times a week, five times a week, six times a week or daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regimen of administration of the enzyme and/or a second agent that is repeated over successive administrations. For example, an exemplary cycle of administration is a 28-day cycle with administration twice weekly for three weeks, followed by one-week of discontinued dosing. A preferred cycle of administration is a 7-day cycle with administration once every 7 days, a 14-day cycle with administration once every 14 days (i.e., 2 weeks), a 21-day cycle with administration once every 21 days (i.e., 3 weeks) or a 28 day cycle with administration once every 28 days (i.e., 4 weeks).

As used herein, when referencing dosage based on mg/kg of the subject, an average human subject is considered to have a mass of about 70 kg-75 kg, such as 70 kg and a body surface area (BSA) of 1.73 m².

As used herein, amelioration of the symptoms of a particular disease or disorder by a treatment, such as by administration of a pharmaceutical composition or other therapeutic, refers to any lessening, whether permanent or temporary, lasting or transient, of the symptoms or, adverse effects of a condition, such as, for example, reduction of adverse effects associated with or that occur upon administration of an B7-H4-targeted antibody-drug conjugate.

As used herein, when referencing dosage based on “body surface area” (BSA; m²) is the measured or calculated surface area of a human body. For many clinical purposes BSA is a better indicator of metabolic mass than body weight because it is less affected by abnormal adipose mass. Various calculations have been published to arrive at the BSA without direct measurement. In the following formulae, BSA is in m², W is mass in kg, and H is height in cm. The most widely used is the Du Bois, Du Bois formula: BSA=0.007184×W^{0.425}×H^{0.725}. Other methods of determining BSA include for example, the Mosteller , Haycock, Gehan and George, Boyd, Fujimoto, Takahira, Shuter and Aslani or Schlich formulas.

A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

As used herein, “unit dose form” or “unit dosage form” refers to physically discrete units suitable for human and animal subjects and packaged individually as is known in the art.

As used herein, a single dosage formulation refers to a formulation as a single dose.

As used herein, “temporal proximity” refers to that administration of one therapeutic agent (e.g., a B7-H4-targeted antibody-drug conjugate disclosed herein) occurs within a time period before or after the administration of another therapeutic agent (e.g., an immune checkpoint inhibitor disclosed herein), such that the therapeutic effect of the one therapeutic agent overlaps with the therapeutic effect of the other therapeutic agent. In some embodiments, the therapeutic effect of the one therapeutic agent completely overlaps with the therapeutic effect of the another therapeutic agent. In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

As used herein a “kit” refers to a combination of components, such as a combination of the compositions herein and another item for a purpose including, but not limited to, reconstitution, activation and instruments/devices for delivery, administration, diagnosis and assessment of a biological activity or property. Kits optionally include instructions of use.

As used herein “prior line of a platinum-containing regimen” include:
i. Adjuvant±neoadjuvant considered as one line of therapy as long as they are the same regimens (e.g., platinum/taxane for 4 cycles before surgery followed by platinum/taxane for 4 cycles after surgery)
ii. Maintenance therapy (e.g., bevacizumab, PARPi or endocrine therapy) will be considered part of the preceding line of therapy. Substitutions of different platinum agents or taxanes will not be counted as new lines.
iii. Therapy given for only 1 cycle and discontinued due to toxicity in the absence of progression will not be counted as a line of therapy. Substitutions of different platinum agents or taxanes will not be counted as new lines.
iv. Hormonal therapy (e.g., tamoxifen, letrozole) will be counted as a separate line of therapy unless it was given as maintenance.

As used herein “recurrent disease” refers to a subject having disease progression following partial or complete response to one or more therapeutics. In some embodiments, the recurrence can be local to the original site of disease (i.e. one or more ovaries) or at a distal or metastatic location.

The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present disclosure is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Other Embodiments

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are illustrative and are not intended to be limiting and it will be readily understood by one of skill in the art that other reagents or methods may be utilized.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.

| Abbreviation | Full Term |
|---|---|
| ADA | Anti-drug antibody |
| ADC | Antibody-drug conjugate |
| AE | Adverse event |
| AF | Auristatin F |
| AF-HPA | Auristatin F bydroxypropyl amide |
| ALT | Alanine transaminase or alanine aminotransferase |
| AST | Aspartate transaminase or aspartate aminotransferase |
| AUC | Area under the curve |
| AUC₀₋ₗₐₛₜ | Area under the concentration curve for the last |
| | measurable concentration |
| BSA | Body surface area |
| Cₘₐₓ | Concentration at maximum level |
| CR | Complete response |
| CTCAE | Common Toxicity Criteria for Adverse Events |
| DAR | Drug to antibody ratio |
| DCR | Disease control rate |
| DES | Dose escalation |
| DL | Dose level |
| DLT | Dose-limiting toxicity |
| DOR | Duration of response |
| EFREAS | Efficacy Response Evaluable Analysis Set |
| ECG | Electrocardiogram |
| ECHO | Echocardiogram |
| EGFR | Epidermal growth factor receptor |
| ER | Estrogen receptor |
| EXP | Expansion |
| HER2 | Human epidermal growth factor receptor 2 |
| HR+ | Hormone receptor positive |
| IHC | immunohistochemistry |
| INR | International normalized ration |
| IRR | Infusion-related reaction |
| HBV | Hepatitis B virus |
| HCV | Hepatitis C virus |
| HIV | Human immunodeficiency virus |
| mAb | Monoclonal antibody |
| MTD | Maximum tolerated dose |
| MUGA | Multi gated acquisition scan |
| nAb | Neutralizing antibody |
| NCI | National Cancer Institute |
| NSAID | Non-steroidal anti-inflammatory drug |
| ORR | Objective response rate |
| OS | Overall survival |
| PD-L1 | Programmed death ligand-1 |
| PFS | Progression-free survival |
| PK | Pharmacokinetics |
| PR | Partial response |
| PT | Prothrombin time |
| q3w | Every 3 weeks dosing |
| Q4W | Every 4 weeks |
| RP2D | Recommended Phase 2 dose |
| RECIST | Response Evaluation Criteria in Solid Tumors |
| SAE | Serious adverse event |
| SAP | Statistical analysis plan |
| SRC | Safety Review Committee |
| t_{1/2} | Terminal elimination |
| tₘₐₓ | Time of maximum observed concentration |
| TNBC | Triple negative breast cancer |
| ULN | Upper limit of normal |
| Vss | Volume of distribution |
| WOCBP | Woman of childbearing potential |

### General Information

The B7-H4-targeted antibody-drug conjugate (e.g. XMT-1660) was prepared as described in U.S. patent application Ser. No. 17/568,376.

CDRs were identified by the Kabat numbering scheme.

The complementarity determining regions (CDR) are defined in accordance to the IMGT numbering system (See IMGT®, the international ImMunoGeneTics information system®. Available online: http://www.imgt.org/).

### Example 1: Study Design and Methodology

The single arm study presented herein is a first-in-human study of a B7-H4-targeted antibody-drug conjugate (XMT-1660) in a Phase 1b, open-label trial in previously treated participants with metastatic TNBC, HR+/HER2− breast cancer, endometrial cancer, or ovarian cancer, fallopian tube cancer, or primary peritoneal cancer. The study is composed of 2 parts: a dose escalation part (DES) and an expansion (EXP) part. The DES part of the study will be the dose finding cohort to assess tolerability and safety of XMT-1660 and to determine the maximum tolerated dose (MTD) and/or recommended phase 2 dose (RP2D). The EXP part of the study will further evaluate the preliminary efficacy and safety of XMT-1660 at the MTD and/or RP2D in participants with advanced/metastatic (1) TNBC; (2) HR+/HER2− breast cancer; and (3) endometrial cancer, ovarian cancer, fallopian tube cancer, or primary peritoneal cancer.

The Bayesian optimal interval (BOIN) design will be used to determine the MTD in the dose escalation portion of the study, with the target DLT rate set at 30%. Seven dose levels are planned to be investigated, but intermediate dose levels can be added based on emerging data. Dose escalations will be done according to the dose escalation scheme in Table 1. Initially, XMT-1660 will be dosed intravenously every 3 weeks, however alternative cycle lengths (e.g., Q4W) may be introduced at the recommendation of the Sponsor and Safety Review Committee based on available data. A single-participant cohort will be utilized for the first dose level and for Dose Level 1.5, if it is opened. All cohorts for subsequent dose levels will have 3 participants.

TABLE 1

| Dose Level | XMT-1660 dose (mg/m²) |
|---|---|
| 1^{a, b} (N = 1) | 7.2 |
| 2 (N = 3) | 14.4 |
| 3 | 21.6 |
| 4 | 28.7 |
| 5 | 38.1 |
| 6 | 50.7 |
| 7 | 67.4 |
| 8 | 87.6 |

^{a}For DL1, if a non-DLT drug-related Grade ≥2 AE (with the exception of Grade 2 nausea or fatigue that resolves within 7 days) is observed during the dose-limiting toxicity (DLT) period, then the dose level for the next cohort will be increased by 50% (DL 1.5) instead of 100% (DL 2).
^{b}A single participant will be enrolled in DLI and DL 1.5, if opened. However, if DLT(s) is observed, 2 additional participants will be added at the same dose level.

Eight dose levels are planned to be investigated but intermediate dose levels can be added based on observed data.

Once the MTD dose is determined, additional participants will be enrolled into the MTD and MTD DL-1 cohorts for a total of approximately 15 participants each cohort.

The SRC will meet throughout the study to discuss ongoing dose escalation. The SRC will review data as outlined in the Charter, including but not limited to safety, PK, and efficacy for the MTD and MTD DL-1 cohorts to determine the RP2D.

Approximately 30 or 35 participants will be enrolled in each cohort. Based on emerging data, the sample size for each cohort may be increased to enroll up to approximately 100 participants.

The RP2D will be determined based on the totality of the clinical data, including safety, preliminary anti-tumor effect, PK, and relevant biomarker data. Thirty or 35 participants will be enrolled in each of the expansion cohort participant populations. Additional participants can be enrolled in each of these populations based on emerging data.

In both the DES and EXP segments, blood sampling will be performed to determine the levels of total antibody (XMT-1604), conjugated and free AF-HPA, and the primary metabolite of AF-HPA (AF). Pharmacokinetic parameters will then be determined from these analytes. Testing for anti-drug antibodies (ADA) and neutralizing antibodies (nAb) will be performed.

Alternative schedules may be evaluated if emerging data suggest that q3w dosing will result in excessive toxicity. If an alternative schedule is evaluated, the starting dose will be the dose for which the maximum tolerated dose (MTD) was not exceeded using the q3w dosing schedule and dose escalation will continue using the guidelines outlined in Table 1.

### Duration of Treatment

Treatment with XMT-1660 dosed intravenously every 3 weeks, may continue indefinitely unless one of the following occurs:
Disease progression
Unacceptable adverse events
Participant withdraws consent for participation from the study
General or specific changes in the participant's condition render the participant unacceptable for further treatment.

### Number of Participants

Eight dose levels are planned to be evaluated in the DES portion of the study, which would require a maximum sample size of 42 participants to determine the MTD. The sample size may be increased, however, based on emerging data and the actual number of dose levels evaluated. Once the MTD is determined, additional participants will be enrolled at the MTD and MTD DL-1 to have approximately 15 participants at each of these dose levels.

In the expansion (EXP) portion of the trial, 30, 35 and 35 participants with advanced/metastatic (1) TNBC; (2) EIR+/HER2− breast cancer; and (3) endometrial cancer, ovarian cancer, fallopian tube cancer, or primary peritoneal cancer, respectively, will be enrolled and be treated at the MTD and/or RP2D determined in the dose escalation portion of the study. Based on emerging data, the sample size for each cancer population can increase to enroll up to approximately 100 participants. The decision to further enroll additional participants will be made based on the totality of data.

Further enrollment with different dosing schedules, biomarker enrichment, or sub-population may be considered. Additional expansion cohorts may also be opened based on emerging data.

### Objectives

The primary and secondary objects for the Dose Escalation and dose Expansion are given below

### Primary Objectives for Dose Escalation

Determine the maximum tolerated dose (MTD) and/or recommended Phase 2 dose (RP2D) of XMT-1660
Assess the safety and tolerability of XMT-1660

### Secondary Objectives for Dose Escalation

Assess the preliminary antitumor activity of XMT-1660

### Primary Objectives for Dose Expansion

Assess further the safety and tolerability of XMT-1660 at MTD and/or RP2D
Assess the preliminary antitumor activity of XMT-1660 at MTD and/or RP2D by measuring ORR and DOR based on RECIST 1.1

### Secondary Objectives for Dose Expansion

Assess the PK of XMT-1660, its release product, and selected metabolites
Assess the development of antidrug antibodies (ADA) and neutralizing antibodies (nAb) to XMT-1660

### Exploratory Objective

Evaluate the tumor expression of B7-H4 and PD-L1 by IHC, as determined by tumor type
Assess the association of level of B7-H4 expression to anti-tumor activity of XMT-1660
Evaluate expression of other markers related to cancer or inflammation in relationship to clinical outcome
Assess the population pharmacokinetic (pop PK) profile of XMT-1660 and determine any pop PK exposure/response relationships

### Eligibility

To be eligible for enrolment in this study, all participants must fulfil all the inclusion criteria and none of the exclusion criteria as defined below:

### Main Criteria for Eligibility

General inclusion criteria for dose escalation and dose expansion are below. Participants will be eligible for the study if all of the following criteria are met:
1. Participant has proven recurrent or advanced solid tumor and has disease progression after treatment with available anti-cancer therapies known to confer benefit or is intolerant to treatment.
2. Participant must be at least 18 years of age.
3. Participant must have an ECOG performance status 0 or 1
4. Participant must be able to understand the study procedures and agree to participate in the study by providing written informed consent.
5. Participant must have cardiac left ventricular ejection fraction (LVEF) ≥50% or ≥the institution's lower limit of normal by either ECHO or MUGA scan.
6. Participants with toxicity from prior therapy or surgical procedures must have recovered to Grade≤1. Participants with alopecia, grade 2 peripheral sensory neuropathy, stable immune-related toxicity such as hypothyroidism on hormone replacement therapy, or adrenal insufficiency treated with ≤10 mg daily prednisone (or equivalent), is an exception to this criterion and may qualify for this study.
7. Participant must have adequate organ function within 14 days prior to cycle 1 day 1 defined by the following criteria:

| | |
|---|---|
| Absolute neutrophil count (ANC), | ≥1500 cells/mm³ |
| not induced by granulocyte | Growth factor prophylaxis cannot be used prior to the first |
| colony-stimulating factors | dose of XMT-1660. |
| Platelet count | ≥100,000/mm³ |
| | Prophylactic transfusion of blood (or blood components) |
| | within 14 days prior to screening cannot be used to meet |
| | enrollment criteria. |
| Hemoglobin | ≥9 g/dL |
| | Prophylactic transfusion of blood (or blood components) |
| | within 14 days prior to screening cannot be used to meet |
| | enrollment criteria. |
| International normalized ratio | ≤1.2 × ULN, all in the absence of any other indicator of |
| (INR)/prothrombin time (PT), | liver dysfunction. |
| and activated partial | Note: Participants on anticoagulants are allowed if their |
| thromboplastin time (aPTT) | INR/PT/PTT is within what is considered to be the |
| | therapeutic range. |
| Estimated glomerular filtration | ≥60 mL/min according to the Chronic Kidney Disease |
| rate (GFR). | Epidemiology Collaboration (CKD-EPI) Creatinine |
| | Equation |
| Total bilirubin | ≤1.2 × ULN |
| Direct bilirubin | ≤ULN |
| | Note: Participants with asymptomatic elevations in direct |
| | bilirubin due to Gilbert syndrome or stable chronic |
| | hemolytic anemia (e.g., hereditary spherocytosis, sickle |
| | cell disease, thalassemia intermedia) may be eligible after |
| | discussion with the study Medical Monitor. |
| Aspartate aminotransferase (AST | ≤1.5 × the institutional ULN |
| or SGOT) and alanine | OR |
| aminotransferase (ALT or SGPT). | ≤2 × the institutional ULN (if known liver metastases) |
| Albumin | ≥3.0 g/dL |

8. During the study, female study participants of child-bearing potential (WOCBP) must use a contraceptive method that is highly effective (with a failure rate <1% per year when used correctly and consistently) during study participation and for 6 months after receipt of the last dose of XMT-1660.
9. Male study participants must use barrier contraception (condoms) and refrain from donating sperm for the duration of study drug treatment and for at least 6 months after the last dose of study drug. Partners of male study participants considered to be WOCBP must use a highly effective contraceptive method for the same duration.
10. Participant must have at least one measurable lesion(s) as defined by RECIST version 1.1.
11. Participant must be willing to undergo a minimally invasive tumor biopsy to obtain tumor tissue for local testing, if medically feasible, prior to C1D1. If the investigator feels a biopsy is not medically feasible, an exemption request must be submitted to the study Medical Monitor for approval. In addition, an archival tissue sample, if available, should be submitted to a central laboratory to enable a longitudinal analysis of tumor biomarkers. Note: Confirmation of expression of B7-H4 is not required for study enrollment.

General exclusion criteria for dose escalation and expansion are below. Participants will not be eligible for the study if any of the following criteria are met:
1. Participant has received prior treatment with another ADC containing an auristatin or maytansinoid payload (e.g., mirvetuxuximab)
   Note: prior treatment with another ADC containing other payloads (non-auristatin or non-maytansinoid) is allowed (e.g., Sacituzumab govitecan, fam-trastuzumab deruxtecan-nxki)
2. Participant has had major surgery within 28 days of starting study treatment, systemic anti-cancer therapy within the time period of 28 days or 5 half-lives of the prior therapy before starting study treatment (14 days or 5 half-lives for small molecule targeted therapy), whichever is less, or palliative radiation therapy within 14 days of starting study treatment
3. Has a diagnosis of additional malignancy that required active treatment (including surgery, systemic therapy, and radiation) within 2 years prior to screening, except for adequately treated basal cell or squamous cell skin cancer, or carcinoma in situ of the breast or of the cervix.
4. Participant has untreated, hepatitis B virus (HBV) or hepatitis C virus (HCV). In addition, negative serology is required during screening (baseline) for HBV and HCV:
   HBV: Participants with HBV infection must have HB s AG (−) and HB c ab (+) serology with viral load below the limit of quantification to be eligible.
   HCV: Participants with a history of HCV infection should have completed curative antiviral treatment and HCV viral load below the limit of quantification.
5. Participants with human immunodeficiency virus (HIV) infection.
6. Participant has a history of cirrhosis, hepatic fibrosis, esophageal or gastric varices, or other clinically significant liver diseases. Liver function testing beyond laboratory studies otherwise defined in the eligibility criteria, to diagnose potentially clinically significant liver disease based on risk factors such as hepatic steatosis or history of excessive alcohol intake, will be based on clinical judgment of the investigator.
7. Participant may not receive a drug associated with hepatotoxicity concurrent with XMT-1660 administration. Participants may receive acetaminophen/paracetamol for a limited time but at a total daily dose of ≤2 g per day. Use of NSAIDs or steroids for treatment of fever is encouraged.
8. Participant has current severe, uncontrolled systemic disease (e.g., clinically significant cardiovascular, pulmonary, or metabolic disease) or intercurrent illness that could interfere with per-protocol evaluations. Further, participants are excluded with the following characteristics:
   A marked baseline prolongation of QT/QTcF interval CTCAE Grade >1: repeated demonstration of a QTcF interval >480 milliseconds (ms) using Fridericia's QT correction formula.
   A history of additional risk factors for Torsades de Pointes (e.g., family history of Long QT Syndrome).
9. Participant is receiving concurrent anti-cancer therapy (e.g., chemotherapy, radiation therapy, biologic therapy, immunotherapy, hormonal therapy, investigational therapy).
10. Participant is unable or unlikely to comply with dosing schedule and study evaluations.
11. Participant has history of or suspected pneumonitis or interstitial lung disease.
12. Participant currently uses either constant or intermittent supplementary oxygen therapy.
13. Participant has oxygen saturation on room air <93%.
14. Participant is using strong CYP450 3A inhibitors or inducers that cannot be discontinued while receiving study treatment. Participants currently on these medications must be discontinued within 14 days of the first dose of study medication.
15. Participant has untreated CNS metastases (including new and progressive brain metastases), history of leptomeningeal metastasis or carcinomatous meningitis.
   a. Participants are eligible if CNS metastases are adequately treated, and participants are neurologically stable for at least 2 weeks prior to enrollment.
   b. In addition, participants must be either off corticosteroids, or on a stable/decreasing dose of ≤10 mg daily prednisone (or equivalent). Anticonvulsants are allowed except for those drugs associated with liver toxicity.

The disease-specific inclusion criteria for dose escalation are outlined in Table 2.

TABLE 2

| Disease | Inclusion criteria |
|---|---|
| HR+/HER2− BC | Received at least one line of systemic therapy which must |
| | have included CDK 4/6 inhibitor + endocrine therapy (ET), |
| | in an advanced or metastatic BC setting |
| TNBC | Received at least 2 lines of systemic therapy in locally |
| | advanced or metastatic BC setting |
| Endometrial Cancer | Received at least 1 line of systemic therapy including |
| | platinum-based chemotherapy for advanced or metastatic |
| | disease |
| Ovarian cancer, Fallopian | Received at least 2 lines of systemic therapy for advanced or |
| Tube cancer, or Primary | metastatic disease, which should include platinum-based |
| Peritoneal Cancer | chemotherapy |

The eligibility criteria for the hormone receptor positive (HR+), human epidermal growth factor receptor 2 negative (HER2−) breast cancer (HR+/HER2− BC) for the dose expansion cohort is:
1. Participant has histologically or cytologically proven diagnosis of breast cancer with evidence of advanced or metastatic disease for whom experimental therapy is appropriate, as determined by the treating physician.
2. Participant has documentation of ER-positive and/or PR-positive breast cancer and HER2-negative tumor based on local testing on most recent tumor biopsy as defined in the relevant American Society of Clinical Oncology (ASCO)/College of American Pathologists (CAP) Guidelines.
3. Participant is required to have received prior therapy with combined CDK 4/6 inhibitor and endocrine therapy in any setting.
4. Participant has not received more than 2 prior lines of chemotherapy for advanced or metastatic disease. For participants with BRCA mutation, treatment with PARP inhibitor is allowed and will not be counted towards chemotherapy.

The eligibility criteria for the triple negative breast cancer (TNBC) dose expansion cohort is:
1. Participant has histologically or cytologically proven diagnosis of breast cancer with evidence of locally advanced or metastatic disease for whom experimental therapy is appropriate, as determined by the treating physician.
2. Participant has documentation of estrogen receptor (ER)-negative, progesterone receptor (PR)-negative and HER2-negative breast cancer based on local testing on most recent tumor biopsy as defined in the relevant American Society of Clinical Oncology (ASCO)/College of American Pathologists (CAP) Guidelines.
3. Participant has received 1-3 prior lines of chemotherapy for metastatic setting. For participants with BRCA mutation, treatment with PARP inhibitor is allowed and will not be counted towards chemotherapy.

There are no disease specific exclusion criteria for either HR+/HER2− BC or TNBC cohorts.

The eligibility criteria for the endometrial cancer dose expansion cohort is:
1. Participant has histologically or cytologically proven diagnosis of endometrial carcinoma, with evidence of metastatic or locally advanced disease who have no satisfactory treatment options and for whom experimental therapy is appropriate, as determined by the treating physician
2. Received at least 1 prior line of platinum-doublet chemotherapy and no more than 3 prior lines of systemic therapy for recurrent or metastatic disease, not including hormonal therapy

The participants to be enrolled in the endometrial cancer cohort must not have endometrial sarcoma or carcinosarcoma.

The inclusion criteria for the ovarian cancer, fallopian tube cancer, or primary peritoneal cancer dose expansion cohort is:
1. Histological diagnosis of high grade serous ovarian cancer, which includes fallopian tube cancer, or primary peritoneal cancer, that is metastatic or recurrent.
2. Participants must have platinum-resistant recurrent disease, defined as completing 4 or more cycles of platinum-based therapy and radiographically progressing within 6 months of last platinum-based therapy.
   a. For participants with 1 prior line of chemotherapy, must have achieved a complete or partial response to that therapy and their disease progressing between 3 and 6 months after the last dose of platinum in the first-line setting.
   b. For participants with 2 to 4 prior lines of chemotherapy, disease progressing within less than or equal to 6 months after the last dose of platinum in the most recent chemotherapy.
3. One to 4 prior lines of systemic therapy for ovarian cancer including at least 1 prior line of a platinum-containing regimen.
   Definitions for prior lines of therapy:
      i. Adjuvant±neoadjuvant is considered one line of therapy as long as they are the same regimens (e.g., platinum/taxane for 4 cycles before surgery followed by platinum/taxane for 4 cycles after surgery)
      ii. Maintenance therapy (e.g., bevacizumab, PARPi or endocrine therapy) will be considered part of the preceding line of therapy. Substitutions of different platinum agents or taxanes will not be counted as new lines.
      iii. Therapy given for only 1 cycle and discontinued due to toxicity in the absence of progression will not be counted as a line of therapy. Substitutions of different platinum agents or taxanes will not be counted as new lines.
      iv. Hormonal therapy (e.g., tamoxifen, letrozole) will be counted as a separate line of therapy unless it was given as maintenance.

The exclusion criteria for the ovarian cancer, fallopian tube cancer, or primary peritoneal cancer dose expansion cohort is:
1. Participants to be enrolled in the ovarian cancer cohort must not have low-grade, clear cell, endometrioid, mucinous, carcinosarcoma, germ-cell, or stromal tumors.
2. Participants to be enrolled in the ovarian cancer cohort must not have primary platinum resistant disease, defined by a lack of response or progression within 3 months after completing front-line, platinum-containing therapy.

### Criteria for Evaluation

### Safety

The frequency and grade of AEs and SAEs based on National Cancer Institute (NCI) Common Terminology Criteria version 5.0 (CTCAE v5.0).

Changes in clinical laboratory parameters (hematology, chemistry, urinalysis), vital signs, ECOG performance status, ECG parameters, physical examinations, and usage of concomitant medications.

### Clinical Activity

Tumor responses (ORR, DOR) and Disease Control Rate (DCR) will be investigator-assessed using Response Evaluation Criteria in Solid Tumors (RECIST), version 1.1 at the end of Cycle 2 and every 2 cycles thereafter.

### Pharmacokinetics

Pharmacokinetics of XMT-1660 (Cₘₐₓ, C_{trough}, tₘₐₓ, AUC, AUCₗₐₛₜ, AUC_{∞}, t½, CL, and Vₛₛ)
In both the DES and EXP segments, blood sampling will be performed to determine the levels of total antibody (XMT-1604), conjugated and free XMT-1267, and primary metabolite of XMT-1267 (XMT-1521). Pharmacokinetic parameters will then be determined from these analytes.
Testing for anti-drug antibodies (ADA) and neutralizing antibodies (nAb) will be performed.
Population pharmacokinetic and population PK exposure/response analysis will also be conducted.

### Dose Limiting Toxicity (DLT)

The DLT observation period is the time between the initiation of Cycle 1 infusion of XMT-1660 and completion of Cycle 1, (e.g. 21 days for Q3W or 28 days for Q4W) and should include the pre-dose assessments before receiving the Cycle 2 dose. Participants must receive at least 80% of their planned dose and complete Cycle 1 in order to be considered evaluable for tolerability, unless dose reduction, interruption, or discontinuation was the result of a DLT.

A DLT is defined as any of the toxicities not due to disease progression or another clearly identifiable cause detailed in the protocol that occurs within the DLT Observation Period.

### Rationale for the Study

Despite advances in novel therapies conferring benefits in progression-free survival (PFS) and overall survival (OS), the majority of patients with unresectable or recurrent/metastatic breast cancer eventually acquire resistance to these therapies and die from their disease. Therefore, unresectable or recurrent/metastatic TNBC and HR+/HER2− breast cancer remains serious and life-threatening with an unmet medical need.

Notably, B7-H4 protein expression has been observed in various human tumors, such as breast, endometrial, and ovarian cancers. In addition, B7-H4 expression in tumor cells of these malignancies appears to be associated with disease progression and poor prognosis. B7-H4 is expressed in ˜77% of TNBC and ˜60% of HR+ and HER2+ tumors. B7-H4 expression was high in ˜45% of breast cancers and was independent of ER/HER2 status. Further, it was reported as broadly expressed in endometrial and ovarian cancers.

The hypothesis is that by targeting B7-H4, the Dolasynthen ADC (XMT-1660) will confer clinical benefit for patients with unresectable or recurrent/metastatic TNBC after standard chemotherapy and HR+/HER2− post-CDK4/6 inhibitor and endocrine-based therapy. To test this hypothesis, the first-in-human study with XMT-1660, a B7-H4-directed antibody conjugated with an auristatin payload, will be initiated in tumors likely to express B7-H4. The primary objective will be to evaluate the safety and tolerability of XMT-1660 as a single agent administered intravenously until disease progression or unacceptable toxicity, in previously treated participants with TNBC, EIR+/HER2− breast cancer, endometrial cancer and ovarian cancer, fallopian tube cancer or primary peritoneal cancer. The secondary objectives will be to obtain pharmacokinetics, immunogenicity, and preliminary efficacy with this dosing regimen. An exploratory objective will be to explore the association of target expression in the tumor and other markers related to cancer or inflammation.

### Example 2: Administration of XMT-1660

The B7-H4-targeted antibody-drug conjugate (e.g. XMT-1660) will be administered according to body surface area (BSA). The BSA adjusted dose will be calculated following each institution's standard practice. When possible, the Mosteller Formula will be used. The starting dose will be calculated based on height and weight collected within 14 days of the first dose (can be collected the day of first dose). Dose calculations in subsequent cycles should be made for weight changes ≥5% from the most recent dose calculation. Dose adjustments based on subsequent weight measurements should be made in accordance with each institution's standard practice. If an institution does not have a standard practice, additional weight measurements will be obtained and BSA confirmed or altered prior to dosing at Cycle 2 and every 2 cycles thereafter.

XMT-1660 is administered IV via antecubital or in-dwelling venous catheter. The initial dose for each participant will be administered over 90 minutes. If no IRR occurs, all subsequent doses can be administered over 60 (±10) minutes at the discretion of the Investigator. Subsequent infusions can be administered on an outpatient basis as long as tolerable, but during Cycles 1-3, participants will be monitored for about 6 hours following XMT-1660 administration for safety assessment and PK sampling.

In the EXP part of the study, once a participant completes Cycle 1, dose adjustments may be made if a participant experiences toxicity but would benefit from further treatment with XMT-1660, in the opinion of the Investigator.

### Example 3: Analysis

The safety analysis set includes all participants receiving any amount of an XMT-1660 dose (partial or complete) in DES and EXP. All safety analyses will be conducted in the Safety Analysis Set, except for the assessment of tolerability. Participants must receive at least 80% of their planned dose of XMT-1660 and complete Cycle 1 to be considered evaluable for tolerability, unless dose reduction, interruption, or discontinuation was the result of a DLT.

The enrolled participants who receive any amount of their assigned dose of XMT-1660 and have an adequate number of concentration determinations to allow for PK calculations will comprise the PK Analysis Set. Analysis of PK concentration data per dose level, per indication, and per participant results will be provided.

The Efficacy Response Evaluable Analysis Set (EFREAS) includes a subset of the efficacy analysis set for whom baseline response assessment and at least one post-baseline response assessment are available.

### Safety Analysis

At the end of the DES phase of the trial, the MTD will be selected as the dose for which the isotonic estimate of the DLT probability is closest to the target DLT rate. The isotonic estimates of the DLT probabilities will be obtained using the pooled adjacent violators algorithm.

The number and percent of participants who experienced DLT by dose level and across all dose levels in the DES phase of the study will be reported.

Participants completing Cycle 1 in the absence of a DLT will be considered to have tolerated the XMT-1660 regimen. The number and percent of participants who tolerated the XMT-1660 regimen will be reported by dose level and across all dose levels in the subset of participants evaluable for assessment of tolerability in the DES phase of the study.

All AEs and adverse events of clinical interest (AECI) will be reported by dose level and across all dose levels in the DES phase of the study. In the expansion phase, all AEs and AECIs will be reported by cancer population. The seriousness, CTCAE severity grading, and Investigator-reported relationship of adverse events to study drug will be summarized and listed. Safety laboratory data will be summarized and listed.

### Primary Efficacy Analysis

The primary analysis of efficacy will be performed using both DES and EXP data. The primary endpoint is ORR by Investigator radiologic review and is defined as the proportion of participants who achieve a confirmed PR or CR per RECIST v1.1. A secondary endpoint is the DCR which is defined as the proportion of participants who achieve complete response, partial response, and/or stable disease of any duration per RECIST v1.1. The number and percentage of participants achieving response or clinical benefit will be summarized and an exact 95% confidence interval (CI) will be provided.

Analysis of other efficacy endpoints including DOR will also be reported, wherever possible according to standard response criteria. Kaplan-Meier estimates of medians and quartiles with 95% CIs will be reported for these statistics. The efficacy endpoints will be analyzed using both the EFAS and the EFREAS in each cancer type cohort.

### Pharmacokinetic Analysis

The PK profiles of the active moieties/release products/metabolites of XMT-1660, will be determined for each participant by non-compartmental analysis using validated PK software (i.e., Phoenix WinNonlin). PK parameters per participant will include time of maximum observed concentration (tₘₐₓ), maximum concentration (Cₘₐₓ), and area under the concentration curve for the last measurable concentration (AUC₀₋ₗₐₛₜ). When the terminal elimination phase can be identified, additional parameters such as terminal elimination (t_{1/2}), clearance (CL), and volume of distribution (Vss) will be determined.

The handling of missing concentration and covariate data, outliers and values below the limit of quantification as well as details of the modeling for dose-response and PK parameter-response relationships will be provided in a separate clinical pharmacology analysis plan.

### Objective Response Rate and Measurement of B7-H4 Expression

B7-H4 expression status will be confirmed by central laboratory testing. ORR and DOR (with associated CIs) will be reported by participants stratified by B7-H4 status.

### Example 4: Tumor Growth Response to Administration of XMT-1660 in CTG-1280 Patient Derived Endometrial Cancer Xenograft Mouse Model

Female athymic nude mice were implanted subcutaneously with CTG-1280 patient derived endometrial cancer tumor fragments. Animals were randomized into treatment groups when tumor volumes were between 168-299 mm³ (mean=221mm³/group) (n=10/group). Vehicle, rituximab isotype control conjugate (prepared as described in US Patent Application No. 2022/0233707A1, 4.60/0.150 mg/kg), XMT-1660 (4.68/0.150 mg/kg, DAR 6) were dosed intravenously on day 1 (all doses are given by antibody/payload). No significant body weight loss or clinical observations were noted.

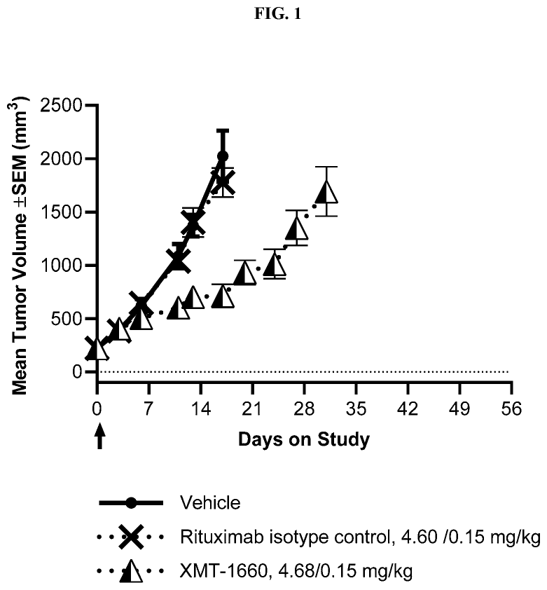

FIG. 1 provides the results for the tumor volumes of CTG-1280 tumor bearing mice treated with isotype control conjugate and XMT-1660. Treatment with isotype control conjugate at 4.60/0.150 mg/kg had no significant effect on tumor volume compared to vehicle control on Day 17 (14% TGI, adjusted p=0.5839). Treatment of XMT-1660 at 4.68/0.150 mg/kg resulted in significantly decreased tumor volume compared to the vehicle control group on Day 17 (73% TGI, adjusted p<0.00001).

### Example 5: Tumor Growth Response to Administration of XMT-1660 in OV2423 Patient Derived Ovarian Cancer Xenograft Mouse Model

Female BALB/c nude mice were implanted subcutaneously with OV2423 patient derived ovarian cancer tumor fragments (˜2-3 mm³ per mouse). Animals were randomized into treatment groups when tumor volumes were between 101-230 mm³ (mean=155mm³/group) (n=8/group). Vehicle, palivizumab isotype control conjugate (prepared as described in US Patent Application No. 2022/0233707A1, 4.54/0.150 mg/kg) and XMT-1660 (4.61/0.150 mg/kg, DAR 6) were dosed intravenously on day 1 (all doses are given by antibody/payload). No significant body weight loss or clinical observations were noted.

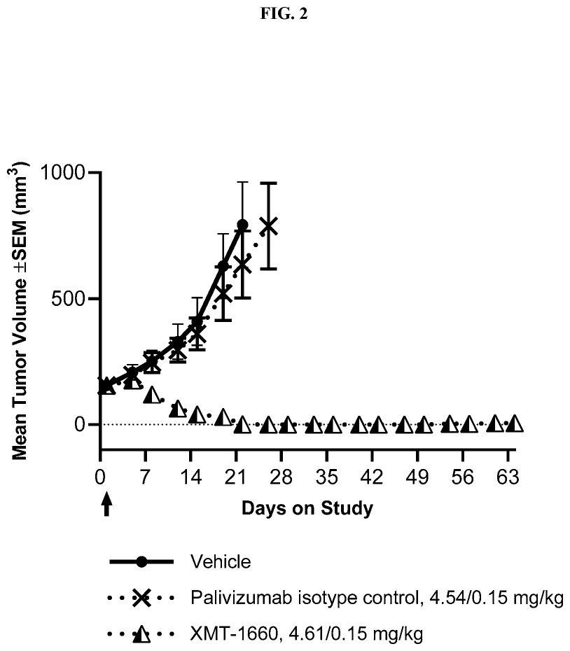

FIG. 2 provides the results for the tumor volumes of OV2423 tumor bearing mice treated with isotype control and XMT-1660. Treatment with palivizumab isotype control conjugate (4.54/0.150 mg/kg) had no significant effect on tumor volume compared to vehicle control on Day 22 (24.8% TGI, adjusted p=0.977). XMT-1660 dosed at 4.61/0.150 mg/kg resulted in significantly decreased tumor volume compared to the vehicle control group on Day 22 (124.05% TGI, adjusted p<0.001) and resulted in 1 PR and 7 TFS.

### Example 6: Tumor Growth Response to Administration of XMT-1660 Cytotoxic Drug Conjugates in CTG-1692 Patient Derived Ovarian Cancer Xenograft Mouse Model

Female athymic nude mice were implanted subcutaneously with CTG-1692 patient derived ovarian cancer tumor fragments. Animals were randomized into treatment groups when tumor volumes were between 131-447 mm³ (mean=229 mm³/group) (n=8/group). Vehicle, palivizumab isotype control conjugate (2.27/0.075 or 4.54/0.150 mg/kg, DAR 6), or XMT-1660 (2.30/0.075 or 4.60/0.150 mg/kg, DAR 6) prepared as described in US Patent Application No. 2022/0233707A1, were dosed intravenously on day 1 (all doses are given by antibody/payload). No significant body weight loss or clinical observations were noted.

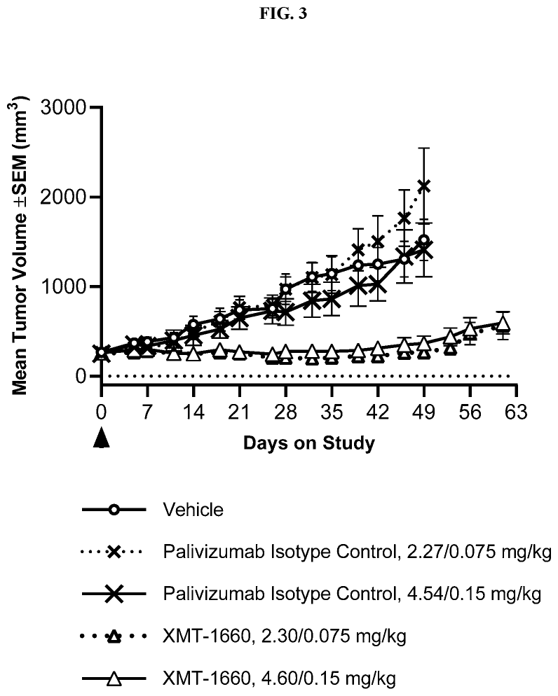

FIG. 3 provides the results for the tumor volumes of CTG-1692 tumor bearing mice treated with isotype control conjugate or XMT-1660. Treatment with isotype control conjugate at 2.27/0.075 or 4.54/0.150 mg/kg had no significant effect on tumor volume compared to vehicle control on day 49 (TGI=−48%, adjusted p=0.2638 or TGI=8%, adjusted p=0.9973, respectively). Treatment with XMT-1660 at 2.30/0.075 mg/kg resulted in significantly decreased tumor volume compared to the vehicle control group on Day 49 (TGI=99%, adjusted p=0.0023) and resulted in 1 PR. Treatment of XMT-1660 at 4.60/0.150 mg/kg resulted in significantly decreased tumor volume compared to the vehicle control group on Day 49 (TGI=92%, adjusted p=0.0058)). Treatment with both dose levels of isotype control conjugate and XMT-1660 at 4.60/0.150 mg/kg resulted in no CRs, PRs, or TFS.

### Example 7: Tumor Growth Response to Administration of XMT-1660 Cytotoxic Drug Conjugates in mBR9013 Syngeneic Breast Cancer Mouse Model

Female FVB/NJ mice were implanted subcutaneously with mBR9013 tumor fragments (2-3 mm³). Animals were randomized into treatment groups when tumor volumes were between 96-277 mm³ (mean=148 mm³/group) (n=8/group). Vehicle, palivizumab isotype control conjugate (1.51/0.050 or 4.54/0.150 mg/kg), XMT-1660 (1.53/0.050 or 4.60/0.150 mg/kg, DAR 6) prepared as described in US Patent Application No. 2022/0233707A1, anti-PD-1 (10.0/0 mg/kg), and XMT-1660+anti-PD-1 (1.53/0.050 mg/kg+10.0/0 mg/kg) were dosed on day 1 (all doses are given by antibody/payload). Vehicle, isotype control conjugate and XMT-1660 were dosed intravenously. Anti-PD-1 was given as an intraperitoneal dose twice a week for 3 weeks, starting on day 1. No significant body weight loss or clinical observations were noted.

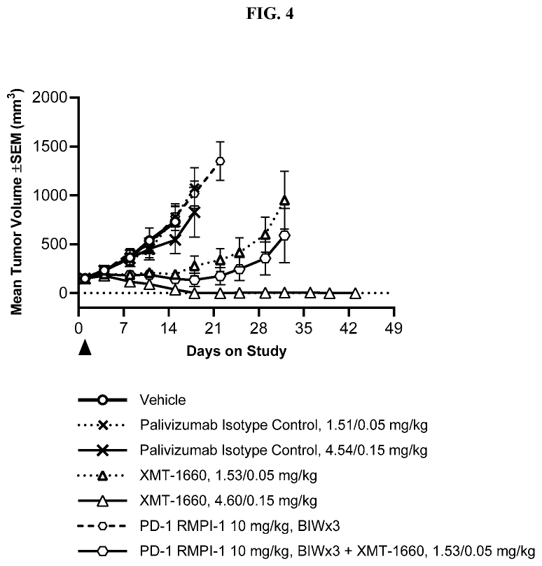

FIG. 4 provides the results for the tumor volumes of mBR9013 tumor bearing mice treated with isotype control conjugate, XMT-1660, and anti-PD-1. Treatment with palivizumab isotype control conjugate at 1.51/0.050 or 4.54/0.150 mg/kg had no significant effect on tumor volume compared to vehicle control on day 15 (TGI=−7.91%, adjusted p=0.999 or TGI=32.02%, adjusted p=0.857, respectively). Treatment of XMT-1660 at 1.53/0.050 or 4.60/0.150 mg/kg resulted in significantly decreased tumor volume compared to the vehicle control group on day 15 (TGI=92.69%, adjusted p=0.000276 or TGI=119.29%, adjusted p=1.45e−08, respectively). Treatment of anti-PD-1 at 10.0/0 mg/kg had no significant effect on tumor volume compared to vehicle control on day 15 (TGI=−9.10%, adjusted p=0.999). Combination treatment of XMT-1660+anti-PD-1 at 1.53/0.050+10.0/0 mg/kg resulted in significantly decreased tumor volume compared to the vehicle control on day 15 (TGI=100.99%, adjusted p=2.34e−05).

### Other Embodiments

While the invention has been described in conjunction with the detailed description thereof, the foregoing description is intended to illustrate and not limit the scope of the invention, which is defined by the scope of the appended claims. Other embodiments, advantages, and modifications are within the scope of the following claims.

1. A method of treating triple negative breast cancer (TNBC), hormone receptor positive/HER2 negative (HR+/HER2−) breast cancer, endometrial cancer, ovarian cancer, fallopian tube cancer, or primary peritoneal cancer in a subject comprising administering to the subject a B7-H4-targeted antibody-drug conjugate by infusion at a dose of between about 7.0 mg/m² to about 90 mg/m²,
wherein the B7-H4-targeted antibody-drug conjugate is a conjugate of Formula (I): 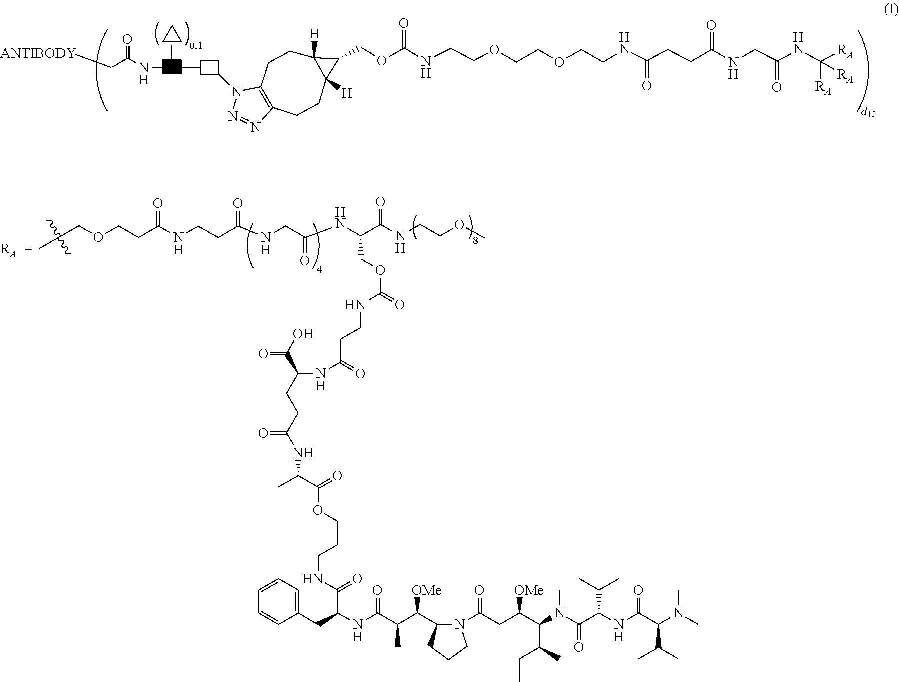
wherein
i. d₁₃ is about 2;
ii. the ANTIBODY binds B7-H4 and comprises a variable heavy chain complementarity determining region 1 (CDRH1) comprising the amino acid sequence GFIVSRNY (SEQ ID NO: 2), a variable heavy chain complementarity determining region 2 (CDRH2) comprising the amino acid sequence IYGSGRT (SEQ ID NO: 3), a variable heavy chain complementarity determining region 3 (CDRH3) comprising the amino acid sequence ARDADYGLDV (SEQ ID NO: 4), a variable light chain complementarity determining region 1 (CDRL1) comprising the amino acid sequence QSVSSSY (SEQ ID NO: 5), a variable light chain complementarity determining region 2 (CDRL2) comprising the amino acid sequence GAS (SEQ ID NO: 6), and a variable light chain complementarity determining region 3 (CDRL3) comprising the amino acid sequence QQYGSSPLYT (SEQ ID NO: 7),
iii. the Drug is attached to a heavy chain of the antibody via a linker moiety at an asparagine residue at position 297 when numbered in accordance with EU numbering; and
iv. ▪ is GlcNAc; Δ is Fuc; and □ is GalNAc.

2. The method of claim 1, wherein the antibody comprises a variable heavy chain comprising the amino acid sequence of SEQ ID NO: 9 and a variable light chain comprising the amino acid sequence of SEQ ID NO: 8.

3. The method claim 1, wherein the antibody comprises a heavy chain comprising the amino acid sequence of SEQ ID NO: 13 and a light chain comprising the amino acid sequence of SEQ ID NO: 12.

4. The method of claim 1, wherein the antibody-drug conjugate dose is:
about 6.7 mg/m² to about 7.7 mg/m²,
about 13.9 mg/m² to about 14.9 mg/m²,
about 21.1 mg/m² to about 22.1 mg/m²,
about 28.2 mg/m² to about 29.2 mg/m²,
about 37.6 mg/m² to about 38.6 mg/m²,
about 50.2 mg/m² to about 51.2 mg/m²,
about 66.9 mg/m² to about 67.9 mg/m², or
about 87.1 mg/m² to about 88.1 mg/m².

5. The method of claim 4, wherein the antibody-drug conjugate dose is about 7.2 mg/m².

6. The method of claim 4, wherein the antibody-drug conjugate dose is about 14.4 mg/m².

7. The method of claim 4, wherein the antibody-drug conjugate dose is about 21.6 mg/m².

8. The method of claim 4, wherein the antibody-drug conjugate dose is about 28.7 mg/m².

9. The method of claim 4, wherein the antibody-drug conjugate dose is about 38.1 mg/m².

10. The method of claim 4, wherein the antibody-drug conjugate dose is about 50.7 mg/m².

11. The method of claim 4, wherein the antibody-drug conjugate dose is about 67.4 mg/m².

12. The method of claim 4, wherein the antibody-drug conjugate dose is about 87.6 mg/m².

13. The method of claim 1, wherein the cancer is B7-H4-positive.

14. The method of claim 1, wherein the subject that has TNBC has received at least 2 lines of systemic therapy in locally advanced or metastatic breast cancer setting.

15. The method of claim 1, wherein the subject that has HR+/HER2− breast cancer post-CDK4/6 inhibitor and endocrine-based therapy has received at least one line of systemic therapy which must have included CDK 4/6 inhibitor and endocrine therapy (ET), in an advanced or metastatic breast cancer setting.

16. The method of claim 1, wherein the subject that has endometrial cancer has received at least 1 line of systemic therapy including platinum-based chemotherapy for advanced or metastatic disease.

17. The method of claim 1, wherein the subject that has ovarian cancer, fallopian tube cancer, or primary peritoneal cancer has received at least 2 lines of systemic therapy for advanced or metastatic disease, which should include platinum-based chemotherapy.

18. The method of claim 1, wherein the subject is human.

19. The method of claim 1, wherein the dose is administered on the first day of treatment and every three weeks thereafter or every four weeks thereafter.

20. The method of claim 19, wherein the dose is administered on the first day of treatment and every three weeks thereafter.

21. The method of claim 19, wherein the dose is administered on the first day of treatment and every four weeks thereafter.

22. The method of claim 4, wherein the dose is administered on the first day of treatment and every three weeks thereafter or every four weeks thereafter.

23. The method of claim 22, wherein the dose is administered on the first day of treatment and every three weeks thereafter.

24. The method of claim 22, wherein the dose is administered on the first day of treatment and every four weeks thereafter.

25. A method of treating triple negative breast cancer (TNBC) in a subject comprising administering to the subject a B7-H4-targeted antibody-drug conjugate by infusion at a dose of between about 7.0 mg/m² to about 90 mg/m²,
wherein the B7-H4-targeted antibody-drug conjugate is a conjugate of Formula (I): 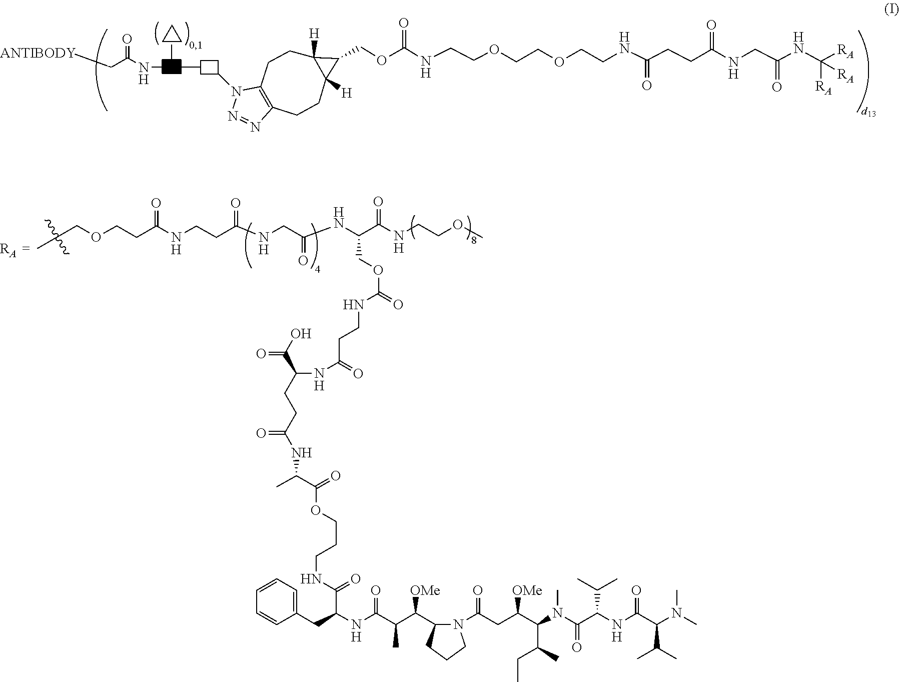
wherein
i. d₁₃ is about 2;
ii. the ANTIBODY binds B7-H4 and comprises a variable heavy chain complementarity determining region 1 (CDRH1) comprising the amino acid sequence GFIVSRNY (SEQ ID NO: 2), a variable heavy chain complementarity determining region 2 (CDRH2) comprising the amino acid sequence IYGSGRT (SEQ ID NO: 3), a variable heavy chain complementarity determining region 3 (CDRH3) comprising the amino acid sequence ARDADYGLDV (SEQ ID NO: 4), a variable light chain complementarity determining region 1 (CDRL1) comprising the amino acid sequence QSVSSSY (SEQ ID NO: 5), a variable light chain complementarity determining region 2 (CDRL2) comprising the amino acid sequence GAS (SEQ ID NO: 6), and a variable light chain complementarity determining region 3 (CDRL3) comprising the amino acid sequence QQYGSSPLYT (SEQ ID NO: 7),
iii. the Drug is attached to a heavy chain of the antibody via a linker moiety at an asparagine residue at position 297 when numbered in accordance with EU numbering; and
iv. ▪ is GlcNAc; Δ is Fuc; and □ is GalNAc.

26. The method of claim 25, wherein the dose is administered on the first day of treatment and every three weeks thereafter.

27. The method of claim 25, wherein the dose is administered on the first day of treatment and every four weeks thereafter.
